(19) United States
(12) Patent Application Publication
(10) Pub. No.: US 2026/0369396 A1
(43) Pub. Date: Nov. 19, 2026

(54) METHODS AND SYSTEMS FOR CONTROLLING MEDICAL DEVICE

(71) Applicant: SHANGHAI UNITED IMAGING HEALTHCARE CO., LTD., Shanghai (CN)

(72) Inventors: Juncheng QIAN, Shanghai (CN); Xiaoyang DUAN, Shanghai (CN); Chunjiang YAO, Shanghai (CN); Shaoting YIN, Shanghai (CN); Yanbang LI, Shanghai (CN); Zhengyang TIAN, Shanghai (CN); Danyi SHEN, Shanghai (CN); Shizhe XU, Shanghai (CN)

(73) Assignee: SHANGHAI UNITED IMAGING HEALTHCARE CO., LTD., Shanghai (CN)

(21) Appl. No.: 19/140,354

(22) PCT Filed: Apr. 28, 2024
(86) PCT No.: PCT/CN2024/090311

§ 371 Date: Jun. 17, 2025

(30) Foreign Application Priority Data
Apr. 28, 2023 (CN) 202310491865.1

(51) Int. Cl. G16H 40/40; G16H 40/63

(52) U.S. Cl. CPC G16H 40/40; G16H 40/63

(57) ABSTRACT

Embodiments of the present disclosure disclose a method for controlling a medical device implemented on a processing device including one or more processors and one or more storage media, comprising: sensing a touch operation via a touch screen, the touch operation being performed by a user on the touch screen; in response to the touch operation, generating, from a plurality of candidate control instructions, at least one target control instruction for controlling the medical device; and for each of the at least one target control instruction, generating, via the touch screen, target sense feedback corresponding to the target control instruction from a plurality types of candidate sense feedback, each of the plurality of candidate control instructions corresponding to one of the plurality types of candidate sense feedback.

### CROSS-REFERENCE TO RELATED APPLICATIONS

This application claims priority to Chinese Patent Application No. 202310491865.1, filed on Apr. 28, 2023, the entire contents of which are hereby incorporated by reference.

### TECHNICAL FIELD

The present disclosure relates to the field of medical technology and, in particular, to methods for controlling a medical device.

### BACKGROUND

With the development of the medical field, various medical devices have been widely used in the medical industry. For medical imaging devices (e.g., CT, MRI, etc.), as a count of patients increases, doctors and technicians, etc., operate the medical imaging devices more and more frequently, but existing methods for controlling the medical imaging devices are based on mechanical buttons, which may easily cause fatigue of the doctors and technicians when the doctors and technicians are operating the medical imaging devices.

Therefore, there is an urgent need to propose methods for controlling medical devices, which can enable a user (e.g., doctors, technicians) to operate the medical device in a natural and labor-saving manner, and improve the interaction experience between the user and the medical device.

### SUMMARY

One or more embodiments of the present disclosure provide a method for controlling a medical device implemented on a processing device including one or more processors and one or more storage media, comprising: sensing a touch operation via a touch screen, the touch operation being performed by a user on the touch screen; in response to the touch operation, generating, from a plurality of candidate control instructions, at least one target control instruction for controlling the medical device; and for each of the at least one target control instruction, generating, via the touch screen, target sense feedback corresponding to the target control instruction from a plurality types of candidate sense feedback, each of the plurality of candidate control instructions corresponding to one of the plurality types of candidate sense feedback.

In some embodiments, the touch operation includes at least one of a single click, a consecutive multi-click, a simultaneous multi-click, sliding from a first position of the touch screen to a second position of the touch screen.

In some embodiments, a real-time comparison between a current value of a touch parameter of the touch operation and a touch threshold on a display interface of the touch screen is displayed.

In some embodiments, a pressure average value is determined based on pressure data of historical touch operations of the user; and a personalized touch threshold corresponding to the user is determined based on the pressure average value.

In some embodiments, the medical device includes a scanning bed, and the at least one target control instruction includes at least one of a start instruction for starting moving the scanning bed, a stop instruction for stopping moving the scanning bed, an instruction for indicating a movement speed of the scanning bed, an instruction for indicating a target position that the scanning bed moves to, or an instruction for indicating a movement direction of the scanning bed.

In some embodiments, the movement speed of the scanning bed is positively correlated with a touch parameter of the touch operation, and the touch parameter is a parameter for describing a touch operation on the touch screen.

In some embodiments, a movement of the scanning bed is controlled based on the at least one target instruction.

In some embodiments, at least one of a movement speed, a position of the scanning bed, or a movement direction of the scanning bed on a display interface of the touch screen during a movement of the scanning bed is displayed in real time.

In some embodiments, the movement of the scanning bed is synchronized by controlling a scanning bed icon in a display interface to move in real time based on the movement of the scanning bed in a physical space.

In some embodiments, the at least one target control instruction is the start instruction for starting moving the scanning bed, and the method further includes: determining whether no touch operation is sensed on the touch screen; in response to determining that no touch operation is sensed on the touch screen, determining whether a touch operation time satisfies a second preset condition; in response to determining that the touch operation time satisfies the second preset condition, controlling the scanning bed to continue moving from an initial moving state; or in response to determining that the touch operation time does not satisfy the second preset condition, controlling the scanning bed to stop moving from the initial moving state.

In some embodiments, a total movement distance of the scanning bed is positively correlated with a touch parameter of the touch operation.

In some embodiments, the total movement distance and/or the target position of the scanning bed corresponding to a current value of the touch parameter is displayed in real time on a display interface of the touch screen.

In some embodiments, the touch operation is performed by a user in a personalized touch region of the touch screen, and the personalized touch region corresponds to the user and a type of the touch operation.

In some embodiments, the personalized touch region is determined based on historical touch operations of the user with the same type as the touch operation.

In some embodiments, the plurality types of candidate sense feedback are different in at least one of a vibration direction, a vibration intensity, a vibration sensation, or a vibration frequency.

In some embodiments, a specified graphical user interface is displayed on the touch screen in response to the touch operation.

In some embodiments, the specified graphical user interface includes: a visual guidance region.

In some embodiments, the visual guidance region includes: a virtual twin device of the medical device and/or at least one control button, and the at least one control button corresponds to the at least one target control instruction.

In some embodiments, the visual guidance region includes: visual feedback of at least one type, the visual feedback of each of the at least one type corresponds to one of the at least one target control instruction.

One or more embodiments of the present disclosure provide a control device of a medical device, comprising: a touch screen for sensing a touch state or a touch-release state of at least one position on the touch screen; a vibration element that is connected with the touch screen and for generating vibrations to generate target sense feedback corresponding to at least one target control instruction; and a processor configured to generate the at least one target control instruction of the medical device in response to the touch state or the touch-release state of the touch screen, and to control the vibration element to generate vibrations.

In some embodiments, the control device is detachably provided or fixedly provided on a housing of the medical device.

In some embodiments, a touch operation in the touch state includes at least one of a single click, a consecutive multi-click, a simultaneous multi-click, sliding from a first position of the touch screen to a second position of the touch screen.

### BRIEF DESCRIPTION OF THE DRAWINGS

This description will be further explained in the form of exemplary embodiments, which will be described in detail by means of accompanying drawings. These embodiments are not restrictive, in which the same numbering indicates the same structure, wherein:

FIG. 1 is a schematic diagram illustrating a system for controlling a medical device according to some embodiments of the present disclosure;

FIG. 2 is an exemplary flowchart illustrating a process for controlling a medical device according to some embodiments of the present disclosure;

FIG. 3A is an exemplary schematic diagram illustrating a touch operation according to some embodiments of the present disclosure;

FIG. 3B is an exemplary schematic diagram illustrating a vibration feedback according to some embodiments of the present disclosure;

FIG. 4A is an exemplary schematic diagram illustrating a display of a specified graphical user interface on a touch screen according to some embodiments of the present disclosure;

FIG. 4B is yet another exemplary schematic diagram illustrating a display of a specified graphical user interface on a touch screen according to some embodiments of the present disclosure;

FIG. 4C is yet another exemplary schematic diagram illustrating a display of a specified graphical user interface on a touch screen according to some embodiments of the present disclosure;

FIG. 5A is an exemplary flowchart illustrating a movement of a scanning bed according to some embodiments of the present disclosure;

FIG. 5B is an exemplary flowchart illustrating a movement of a scanning bed according to some embodiments of the present disclosure;

FIG. 5C is an exemplary schematic diagram illustrating a process for determining a movement direction of a scanning bed according to some embodiments of the present disclosure;

FIG. 6 is a first exemplary schematic diagram illustrating a touch operation according to some embodiments of the present disclosure;

FIG. 7 is a second exemplary schematic diagram illustrating a touch operation according to some embodiments of the present disclosure;

FIG. 8 is a third exemplary schematic diagram illustrating a touch operation according to some embodiments of the present disclosure;

FIG. 9 is a fourth exemplary schematic diagram illustrating a touch operation according to some embodiments of the present disclosure;

FIG. 10 is a fifth exemplary schematic diagram illustrating a touch operation according to some embodiments of the present disclosure;

FIG. 11 is an exemplary schematic diagram illustrating a process for generating at least one target control instruction according to some embodiments of the present disclosure;

FIG. 12 is an exemplary schematic diagram illustrating a process for determining a touch operation time according to some embodiments of the present disclosure; and

FIG. 13 is an exemplary module diagram illustrating a system for controlling a medical device according to some embodiments of the present disclosure.

### DETAILED DESCRIPTION

The technical schemes of embodiments of the present disclosure will be more clearly described below, and the accompanying drawings need to be configured in the description of the embodiments will be briefly described below. Obviously, the drawings in the following description are merely some examples or embodiments of the present disclosure, and will be applied to other similar scenarios according to these accompanying drawings without paying creative labor. Unless obviously obtained from the context or the context illustrates otherwise, the same numeral in the drawings refers to the same structure or operation.

It should be understood that the “system,” “device,” “unit,” and/or “module” used herein is a method for distinguishing different components, elements, components, parts or assemblies of different levels. However, if other words may achieve the same purpose, the words may be replaced by other expressions.

As shown in the present disclosure and claims, unless the context clearly prompts the exception, “a,” “one,” and/or “the” is not specifically singular, and the plural may be included. It will be further understood that the terms “comprise,” “comprises,” and/or “comprising,” “include,” “includes,” and/or “including,” when used in present disclosure, specify the presence of stated features, integers, steps, operations, elements, and/or components, but do not preclude the presence or addition of one or more other features, integers, steps, operations, elements, components, and/or groups thereof.

The flowcharts are used in present disclosure to illustrate the operations performed by the system according to the embodiment of the present disclosure. It should be understood that the preceding or following operations is not necessarily performed in order to accurately. Instead, the operations may be processed in reverse order or simultaneously. Moreover, one or more other operations may be added to the flowcharts. One or more operations may be removed from the flowcharts.

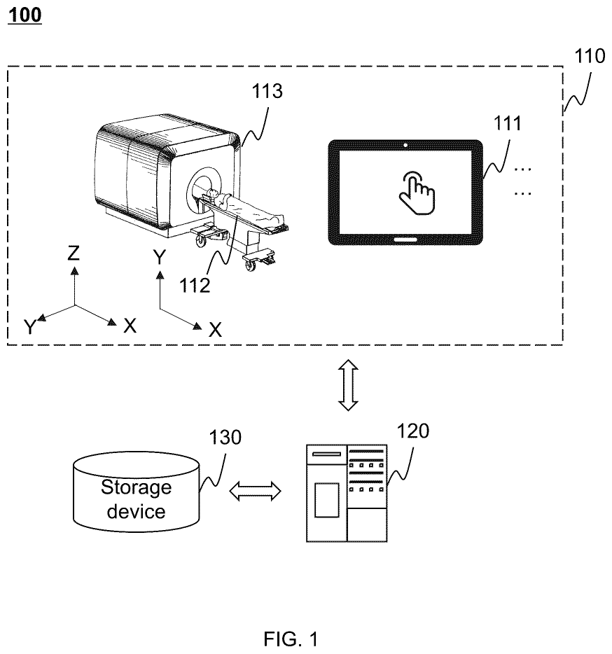

FIG. 1 is a schematic diagram illustrating a system for controlling a medical device according to some embodiments of the present disclosure.

In some embodiments, the system 100 for controlling a medical device may include a medical device 110, a processor 120, and a storage device 130.

The medical device 110 refers to a medical device that is configured to perform functions such as examining or treating a subject (e.g., a patient). In some embodiments, the medical device 110 may include an imaging device, such as a single modality imaging device or a multi-modality imaging device. The single modality imaging device may include an X-ray device, a computed tomography imaging device (CT), a three-dimensional (3D) CT, a four-dimensional (4D) CT, an ultrasound imaging device, a fluorescence fluoroscopy imaging device, a magnetic resonance imaging (MRI) device, a single-photon emission computed tomography (SPECT) device, a positron emission tomography (PET) device, etc. The multi-modality imaging device may include an MRI-CT imaging device, an MRI-PET imaging device, an MRI-SPECT imaging device, a DSA-MRI imaging device, a CT-PET imaging device, a CT-SPECT imaging device, etc. The imaging devices provided above are for illustrative purposes only and are not intended to limit the scope of the present disclosure. As used herein, the term “modality” broadly refers to an imaging method or technique that collects, generates, processes, and/or analyzes imaging information of a target object.

In some embodiments, the medical device 110 includes a treatment device, the medical device being configured to perform a radio therapy on a subject. For example, the treatment device may include a linear accelerator (an accelerator of species of particles including, for example, photons, electrons, protons, or heavy ions), a cyclotron, a synchrotron, etc., configured to perform the radio therapy on the subject.

In some embodiments, the medical device 110 may include both an imaging device and a treatment device, to perform an image guided radiotherapy (IGRT), i.e., a technique used to perform a radiotherapy guided (assisted) by an image.

In some embodiments, the system 100 may include at least one touch screen 111. For example, the touch screen 111 may be a part of the medical device 110. The touch screen 111 may be detachably provided or fixedly provided in the medical device 110. As another example, the touch screen 111 may be a standalone device that may be detached from the medical device 110. As another example, the touch screen 111 may be a component of the medical device 110 and be an integral device with the medical device 110. In some embodiments, the touch screen 111 may also be integrated with the processor 120 as a single device.

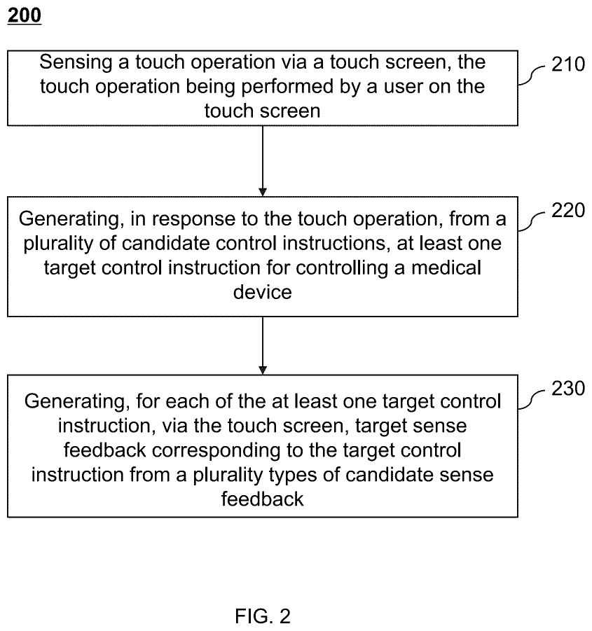

The touch screen 111 refers to a display device that may sense a touch operation of a user. The touch screen 111 may include a touch detection device, a touch screen controller, and a sense feedback element. The touch detection device is configured to detect touch information of the touch operation of the user, such as a touch region of the touch operation, a touch pressure, a number of fingers, a touch distance, a touch duration, a click interval, etc., and then transmit the touch information to the processor 120. For example, the touch screen 111 has a built-in pressure sensing device and senses the touch operation of the user by the pressure sensing device. The sense feedback element may include a voice feedback element, a vibration element, etc. The vibration element may be a vibration motor, etc., which is capable of vibrating and acting on a localized or total region of the touch screen 111, thereby enabling the touch screen 111 to generate sense feedback in the touch region. For example, when the user touches the touch screen, the touch operation may be detected by the touch detection device. Touch information corresponding to the touch operation may be fed back to the processor 120. In such a case, the processor 120 may be configured to determine a corresponding touch region based on the received touch information and control the vibration element corresponding to the touch region to generate vibration feedback to achieve the effect of vibration in the localized region. For more information about the touch operation, the touch region, and the sense feedback, please refer to FIG. 2 and its related descriptions.

In some embodiments, the touch screen 111 may present a display interface to the user. The display interface is configured to enable an interaction between the user and the medical device 110. In some embodiments, the display interface may display, in real time, a movement speed corresponding to a touch parameter. In some embodiments, the display interface may also display, in real time, a position of the scanning bed on the display interface of the touch screen during a movement of the scanning bed. In some embodiments, the display interface may also display, in real time, a total movement distance of the scanning bed corresponding to the touch parameter and/or a target position of the scanning bed. For more information about the display interface, please refer to its related description below.

In some embodiments, the medical device 110 may also include at least one of a scanning bed 112, a scanning device 113, etc. A patient may be disposed on the scanning bed 112. By moving the scanning bed 112, the patient may be moved to a designated location, and the medical device 110 performs a test or treatment, etc., on the patient via the scanning device 113.

The processor 120 may process data and/or information obtained from the medical device 110 (e.g., the touch screen 111, etc.) and/or a storage device 130. In some embodiments, the processor 120 may process the touch operation sensed by the touch screen 111 to generate at least one target control instruction for controlling the medical device 110. For more information about the target control instruction, please refer to FIG. 2 and its related description.

In some embodiments, the processor 120 may include one or more processing engines (e.g., a single-chip processing engine or a multi-chip processing engine). By way of example only, the processor 120 may include a central processing unit (CPU), a graphics processing unit (GPU), a digital signal processor (DSP), a microprocessor, etc., or any combination of the above.

In some embodiments, the processor 120 may be a part of the medical device 110 or the touch screen 111. For example, the processor 120 may be integrated into the medical device 110 or the touch screen 111.

The storage device 130 may store data, instructions (e.g., a control instruction, etc.), and/or any other information. In some embodiments, the storage device 130 may store data obtained from the touch screen 111 and/or the processor 120. In some embodiments, the storage device 130 may store data and/or instructions that the processor 120 may execute or use to perform exemplary methods described in the present disclosure.

In some embodiments, the storage device 130 may be a part of the processor 120 or may be separate and directly or indirectly connected to the processor 120.

In some embodiments, the storage device 150 may include a mass storage device, a removable storage device, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage device 150 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the system 100 for controlling a medical device may further include a network. The network may include any suitable network that can facilitate the exchange of information and/or data for the system 100 for controlling a medical device. In some embodiments, one or more components of the system 100 for controlling a medical device (e.g., the touch screen 111, the scanning bed 112, the scanning device 113, the processor 120, or the storage device 130) may communicate information and/or data with one or more other components of the system 100 for controlling a medical device via the network. For example, the processing device 120 may obtain touch information of the touch operation of the user from the touch screen 111 via the network. In some embodiments, the network may be any type of wired or wireless network, or a combination thereof. The network may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (“VPN”), a satellite network, a telephone network, routers, hubs, switches, server computers, and/or any combination thereof. Merely by way of example, the network may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network may include one or more network access points. For example, the network may include wired and/or wireless network access points such as base stations and/or internet exchange points through which one or more components of the system 100 for controlling a medical device may be connected to the network to exchange data and/or information.

It should be noted that the system is provided for illustrative purposes only and is not intended to limit the scope of the present disclosure. A variety of modifications or variations may be made for those skilled in the art based on the description of the present disclosure. For example, the system may also include a database. As another example, the system may be implemented on other devices to achieve similar or different functionality. However, the changes and modifications will not depart from the scope of the present disclosure.

FIG. 2 is an exemplary flowchart illustrating a process for controlling a medical device according to some embodiments of the present disclosure. In some embodiments, process 200 may be performed by the processor 120.

Step 210, the processor 120 is configured to sense a touch operation via a touch screen (e.g., the touch screen 111 in FIG. 1), the touch operation being performed by a user on the touch screen.

For more information about the touch screen, please refer to FIG. 1 and its related description.

The touch operation refers to a touch action performed by the user on the touch screen. For example, the touch operation includes an operation performed by the user on the touch screen by means of a finger or other touch device, such as a click operation, a slide operation, etc. The click operation refers to an operation performed by the user at a position on the touch screen. The sliding operation refers to an operation performed by the user on the touch screen by sliding from one position to another. For example, the touch device includes, but is not limited to, a stylus or the like. It should be noted that the present disclosure is illustrated with a user's finger touch as an example, and it can be understood that the present disclosure may also be applicable to other touch devices.

In some embodiments, based on the pressure-sensing technology, when the user touches the touch screen 111, the processor 120 may obtain the touch operation of the user through the touch screen 111 and detect the touch operation of the user. For example, the processor 120 may detect a touch region, a touch pressure, a number (or count) of touch fingers of the user, etc.

In some embodiments, the touch operation may include a slide operation. In some embodiments, the slide operation may include a gesture sliding from a first position of the touch screen 111 to a second position of the touch screen 111. The sliding from the first position to the second position is continuous, i.e., the finger always touches the touch screen 111 during the sliding from the first position to the second position. A path from the first position to the second position may be a straight line or a curve. In some embodiments, the slide operation may be a single-finger sliding operation. The single-finger sliding operation refers to performing the gesture sliding from the first position of the touch screen 111 to the second position of the touch screen 111 with one finger. In some embodiments, the slide operation may include a simultaneous multi-finger sliding operation. The simultaneous multi-finger sliding operation is a separate gesture of sliding performed by each of two or more fingers, and at least a portion of the sliding process of each finger is performed at the same time.

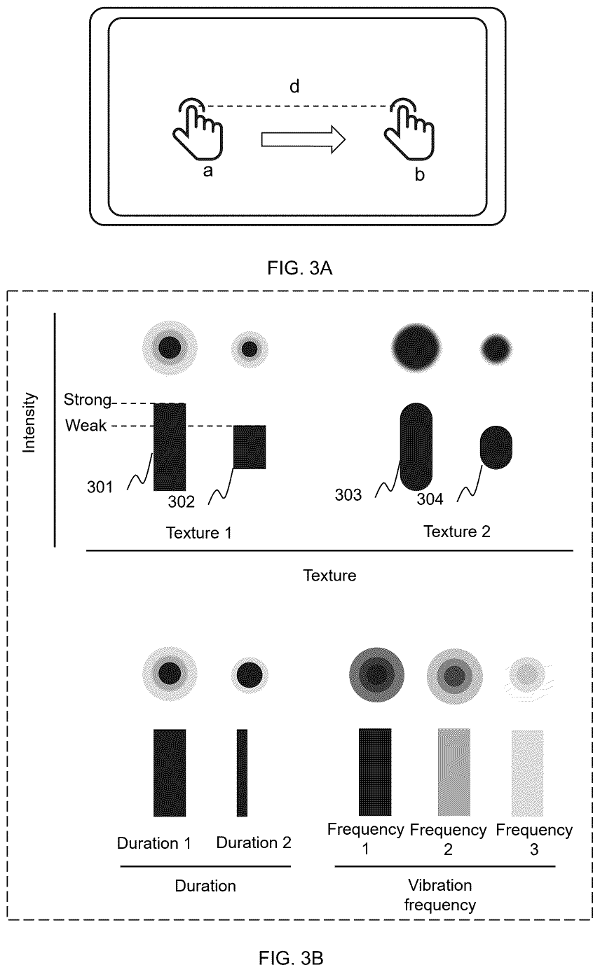

The first position and the second position may refer to two different positions during the touch operation of the touch screen 111 by the user. For example, the first position may refer to a start position when the user performs a certain touch operation on the touch screen 111, and the second position may refer to an end position when the user performs the certain touch operation on the touch screen 111. FIG. 3A is an exemplary schematic diagram illustrating a touch operation according to some embodiments of the present disclosure. As shown in FIG. 3A, the touch operation is a user sliding to operate a position a of the touch screen 111 and sliding to a position b. The position a is the first position and the position b is the second position.

In some embodiments, the touch operation may include a click operation. The click operation may include one or more of a plurality of operations such as a single click, a simultaneous multi-click, a consecutive multi-click, etc. The single click refers to an action of clicking the touch screen with one finger. The simultaneous multi-click refers to an action of clicking the touch screen with at least two fingers simultaneously. The consecutive multi-click refers to an action of consecutively clicking the touch screen at least twice with one finger. The single click may include a light click, a heavy click, etc. The light click and the heavy click correspond to different touch pressures. The light click and the heavy click refer to touch actions of different strengths applied by the finger to the touch screen when performing the touch operation. The light click refers to the finger gently touching the screen and then quickly lifting up, without applying too much pressure or without stay too long click operation. The heavy click refers to the finger touching the screen more forcefully, applying greater pressure to the click operation than the light click. The single click, and the consecutive multi-click correspond to different numbers of touches. The processor 120 may determine what kind of touch operation is performed based on whether a time interval between multiple intermittent touching of the touch screen 111 by the user is less than an interval threshold. For example, if the user intermittently touches the touch screen 111 at least twice, and the time interval is less than the interval threshold, then the user's touch operation is the consecutive multi-click. For example, the user intermittently touches the touch screen 111 twice, and the time interval is greater than the interval threshold, then the user's touch operation is single click.

In some embodiments, the touch operation may correspond to a touch parameter (e.g., a touch distance, a touch pressure, a number of touch fingers, a touch duration) for characterizing the touch operation. For example, a touch parameter of the slide operation may include a touch distance, a touch pressure, a number of touch fingers, etc. The touch distance refers to a straight line distance between the first position and the second position, or a length of a sliding path between the first position and the second position. The touch pressure of the slide operation characterizes a strength level of finger pressure on the touch screen 111 during the process of sliding from the first position to the second position. The touch pressure of the slide operation may refer to an average pressure, a maximum pressure, a minimum pressure, a real-time pressure, or a pressure at a specific position (e.g., the first position, the second position, a midpoint of the sliding path, etc.) of the finger pressing on the touch screen 111 during the process of sliding from the first position to the second position, etc. As another example, a touch parameter of the click operation may include a number of touching fingers, a touch pressure, a touch duration of a single click of each finger of the touch screen, an interval between two adjacent clicks of each finger of the touch screen, etc. The touch pressure of the click operation characterizes a strength level of finger pressure in the process of a single click on the touch screen, which may refer to an average pressure, a maximum pressure, a minimum pressure, a real-time pressure, etc., of a single finger in the process of a single click on a certain position of the touch screen.

In some embodiments, different touch operations may correspond to different target control instructions.

In some embodiments, different touch operations refer to touch operations of different kinds and/or with different touch parameters. For example, the slide operation and the click operation belong to different kinds of touch operations. The single click, the consecutive multi-click, and the simultaneous multi-click belong to different kinds of touch operations. The slide operation with different touch distances also belongs to different kinds of touch operations.

In some embodiments, the touch operation and the target control instruction may have one-to-one correspondence, or a touch operation may generate two or more target control instructions simultaneously.

In some embodiments, different touch operations may be capable of forming different target control instructions. Corresponding to the different target control instructions, tactile, visual, and/or auditory effects may be generated by combinations of vibration, sound, and/or light to provide feedback to the user.

In some embodiments of the present disclosure, multiple target control instructions may be generated by multiple touch operations, which is conducive to improving the interaction experience between the user and a medical device (e.g., the medical device 110 in FIG. 1).

Step 220, in response to the touch operation, the processor is configured to generate, from a plurality of candidate control instructions, at least one target control instruction for controlling the medical device.

For more information about the medical device 110, please refer to FIG. 1 and its related description.

The plurality of candidate control instructions may refer to possible instructions for controlling the medical device. The at least one target control instruction may be selected from the plurality of candidate control instructions. The at least one target control instruction is an instruction that controls the medical device 110 to perform a corresponding action. For example, the at least one target control instruction controls the medical device 110 to turn on. As another example, the at least one target control instruction controls the medical device 110 to stop working. As another example, the at least one target control instruction controls the medical device 110 to move, etc.

In some embodiments, the processor 120 may automatically generate the plurality of candidate control instructions and touch operations corresponding to the plurality of candidate control instructions in advance and storing the plurality of candidate control instructions and touch operations in the storage device 130. For example, the processor 120 may automatically generate the plurality of candidate control instructions and touch operations corresponding to plurality of candidate control instructions thereof based on a type of medical device. Exemplarily, the processor 120 automatically generates a candidate control instruction of “turning on the medical device”, and a corresponding touch operation of “clicking the touch screen”.

In some embodiments, the user may customize the generation of the plurality of candidate control instructions and the corresponding touch operations, and store the plurality of candidate control instructions and touch operations in the storage device 130. For example, the processor 120 may customize a candidate control instruction of “turning on the medical device” to correspond to the touch operation of “clicking the touch screen” based on user's personal operating habits.

In some embodiments, in response to the touch operation, the processor 120 may, based on the touch operation, obtain a candidate control instruction corresponding to the touch operation from the storage device 130, designate the candidate control instruction as a target control instruction, and control an action such as movement of the medical device 110.

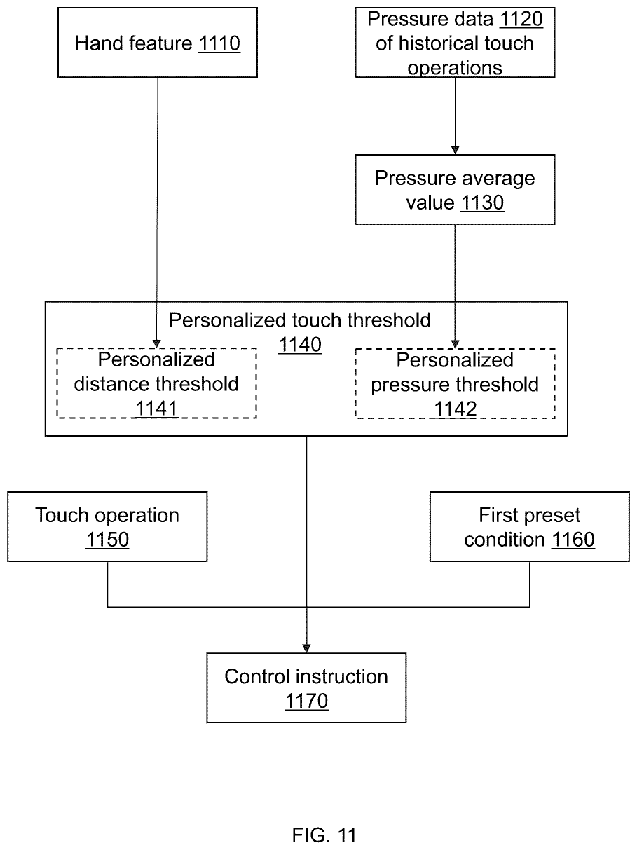

In some embodiments, the processor 120 may obtain a touch threshold and generate a target control instruction when the touch operation satisfies a first preset condition. The first preset condition is that the touch parameter of the touch operation is greater than the touch threshold, and for more information, please refer to FIG. 11 and its related description.

In some embodiments, the medical device 110 may include a scanning bed (e.g., the scanning bed 112). The target control instruction may include at least one of a start instruction for starting moving the scanning bed, a stop instruction for stopping moving the scanning bed, an instruction for indicating a movement speed of the scanning bed, an instruction for indicating a target position that the scanning bed moves to, or an instruction indicating a movement direction of the scanning bed. For more information about the scanning bed 112, please refer to FIG. 1 and its related description.

In some embodiments, the instruction for indicating the movement direction of the scanning bed is configured to determine a movement direction of the scanning bed 112.

In some embodiments, the movement direction of the scanning bed 112 includes a horizontal direction and/or a vertical direction. The horizontal direction may include a direction for moving the scanning bed into a gantry (also referred to as “in-bed direction”) and/or a direction for moving the scanning bed 112 out of the gantry (also referred to as “out-bed direction”). A movement direction close to the scanning device 113 is the direction for moving the scanning bed into the gantry. A movement direction away from the scanning device 113 is the direction for moving the scanning bed 112 out of the gantry. The vertical direction may include a direction for raising the scanning bed 112 (also referred to as “raised-bed direction”) and/or a direction for lowering the scanning bed 112 (also referred to as “lowered-bed direction”). A movement direction away from the ground is the direction for raising the scanning bed 112. A movement direction near the ground is the direction for lowering the scanning bed 112.

In some embodiments, the instruction indicating the movement direction of the scanning bed may be determined based on a touch distance of the user satisfying a third preset condition.

The touch distance of the user is a touch distance of the user's finger. For example, a distance between the user's finger sliding from the first position of the touch screen 111 to the second position of the touch screen 111. As shown in FIG. 3A, a distance d between the user's finger sliding from a position a of the touch screen 111 to a position b of the touch screen 111 is the touch distance of the user.

The third preset condition refers to a preset condition to be satisfied by the touch distance when determining the instruction indicating the movement direction. For example, the third preset condition may include that a number of fingers of the user performing the touch operation is 1, and that the sliding direction satisfies a preset angle range, and that the touch distance of the user is within a distance range (including greater than a distance threshold). The distance threshold may be set according to actual needs, for example, the distance threshold is 3 cm, etc. The third preset condition is that the user's touch distance is greater than the distance threshold value of 3 cm. The preset angle range may be a system default value, a system preset value, etc.

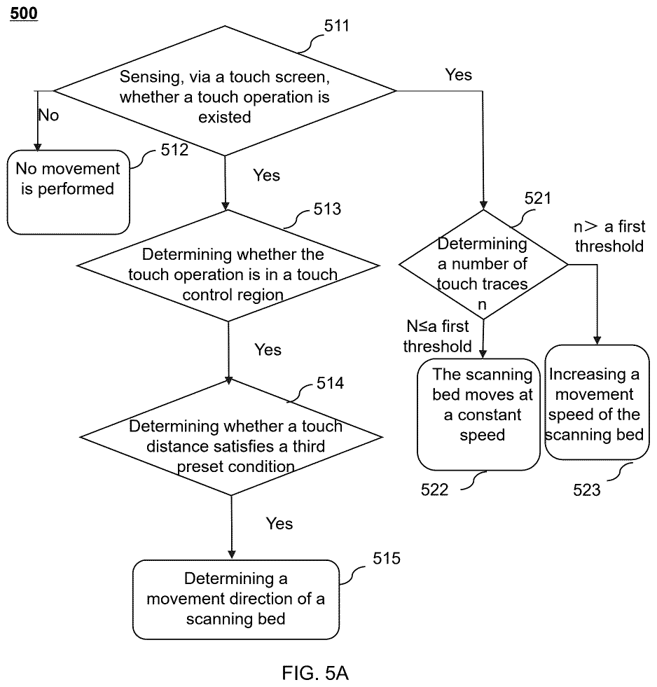

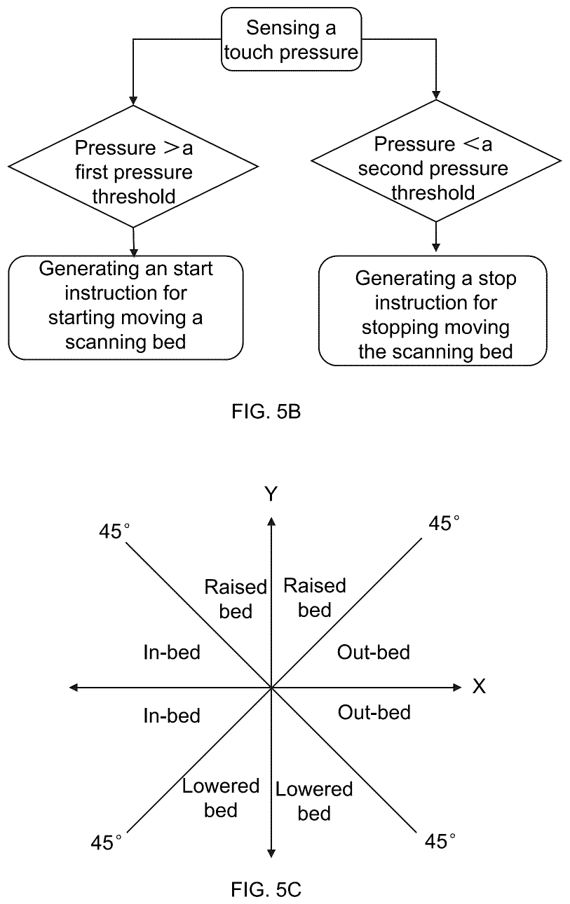

When the processor 120 senses via the touch screen 111 the touch operation of the user, the processor 120 may determine whether the touch distance of the user satisfies the third preset condition. In response to determining that the touch distance of the user satisfies the third preset condition, the processor 120 may determine a movement direction of the scanning bed 112 based on a direction from the first position to the second position, and generate an instruction indicating the movement direction of the scanning bed. For example, as shown in FIG. 5C, FIG. 5C is an exemplary schematic diagram illustrating a process for determining a movement direction of a scanning bed, according to some embodiments of the present disclosure. The movement direction of the scanning bed 112 (an in-bed direction, an out-bed direction, a raised bed direction, and a lowered bed direction) is divided into four regions that are equidistant from the XY axis by 45°. The directions from the first position to the second position are located in different regions corresponding to different movement directions of the scanning bed. For example, in FIG. 3A, a direction from position a to position b is located in a region of moving the scanning bed 112 out of the gantry, and a movement direction of a corresponding scanning bed 112 is determined to be the out-bed direction. A bed-out instruction of the scanning bed is generated.

In some embodiments, the X-axis is a horizontal direction on the touch screen and the Y-axis is a vertical direction on the touch screen. For example, when the user operates the touch screen horizontally, a long side of the touch screen is oriented in the horizontal direction, and a short side of the touch screen is oriented in the vertical direction. For example, when the user operates the touch screen in the vertical direction, the short side of the touch screen is in the horizontal direction and the long side is in the vertical direction. The four regions that are equidistant from the XY axis by 45° refer to regions formed by two 45° straight lines that separate the regions in the first and third quadrants from the regions in the second and fourth quadrants. For example, the region located between the first and second quadrants is the region bounded by the line y=x and the line y=−x.

In some embodiments, after the processor 120 senses via the touch screen 111 the touch operation of the user, at least one candidate movement direction of the scanning bed 112 may be displayed based on the touch screen 111, and an instruction indicating a movement direction of the scanning bed may be determined based on a touch distance of the user in at least one candidate movement direction satisfying the third preset condition.

The candidate movement direction may refer to a direction to be determined as a movement direction of the scanning bed 112. The candidate movement direction may be used by the user to refer to a correspondence between a sliding direction of the finger and the movement direction of the bed. The candidate movement direction may be determined based on a preset rule or the like. The preset rule may refer to a rule related to the touch operation and the generation of the candidate movement direction. For example, the preset rule may be that when the user touches the screen (e.g., when the finger is placed in the first position), the processor may display four regions in the first position of the touch screen (the first position may be used as the origin) as shown in FIG. 5C, and each of the four regions correspond to a candidate movement direction (in-bed direction, out-bed direction, raised bed direction, and lowered bed direction of the scanning bed) for the user's reference. The display as shown in FIG. 5C disappears when the instruction indicating the movement direction is triggered successfully. In some embodiments, the four regions as shown in FIG. 5C may simply be built into a processing logic of the processor 120, which is equivalent to the user blindly operating on the touch screen.

In some embodiments, when the processor 120 determines that a sliding touch distance of the user in a candidate movement direction satisfies the third preset condition, the processor 120 may determine the candidate movement direction as a movement direction of the scanning bed 112, and generate an instruction indicating the movement direction of the scanning bed.

In some embodiments, the start instruction for starting moving the scanning bed is configured to control the scanning bed 112 to move in a certain movement direction.

In some embodiments, the start instruction for starting moving the scanning bed is determined based on a touch pressure (e.g., a touch pressure of a single-finger slide operation or a touch pressure of a single-click operation) obtained by the touch screen 111 satisfying a fourth preset condition.

The fourth preset condition is that the touch pressure is within a pressure range, e.g., greater than a pressure threshold. For example, if the pressure threshold is 3N, the fourth preset condition is that the touch pressure is greater than 3N.

When the touch pressure obtained by the touch screen 111 satisfies the fourth preset condition, the processor 120 may generate a start instruction for starting moving the scanning bed to control the movement of the scanning bed 112.

In some embodiments, a sequence of the generation of the start instruction for starting moving the scanning bed and the instruction for indicating a movement direction of the scanning bed may or may not be sequential.

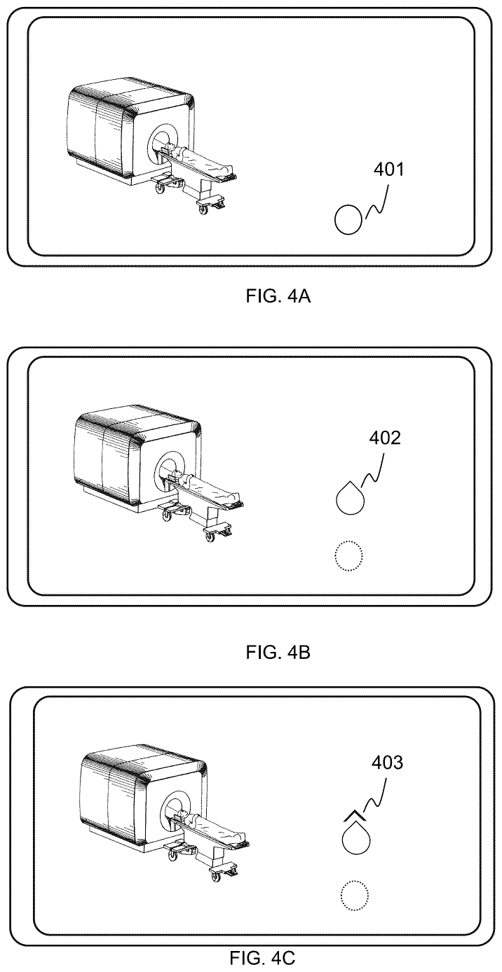

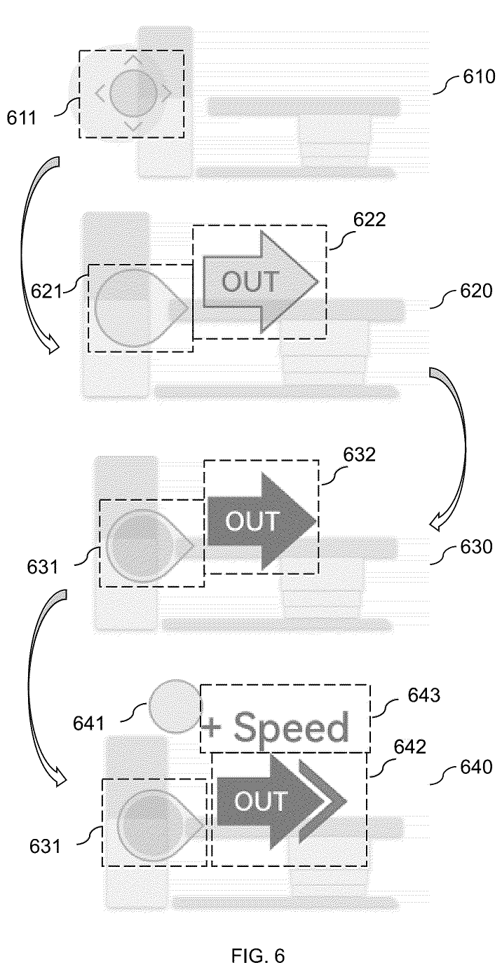

In some embodiments, the start instruction for starting moving the scanning bed may be determined after the instruction for indicating a movement direction of the scanning bed has been determined. For example, the user touches the first position of the touch screen 111 with a touch pressure that does not satisfy the fourth preset condition and slides to the second position with a touch distance that satisfies the third preset condition, thereby determining the instruction for indicating a movement direction of the scanning bed, and increases the touch pressure at the second position until it satisfies the fourth preset condition, thereby determining the start instruction for starting moving the scanning bed. In some embodiments, in response to different touch operations, different indication markers may be displayed on the touch screen. Different indication markers may indicate different scanning directions. For example, as shown in FIG. 6 at 610, firstly, when the user touches the touch screen (the finger does not leave the touch screen and does not satisfy the fourth preset condition), an indication marker is displayed as a circle 611; secondly, the user slides in the out-bed (OUT) direction, as shown in FIG. 6 in 620, and when the touch distance of the user satisfies the third preset condition, an indication marker is displayed as an irregular shape 621 having an arrow and an arrow 622 pointing to the out-bed (OUT) of the movement direction of the scanning bed; thirdly, the user presses down on the touch screen, as shown in FIG. 6 at 630, and when the touch pressure satisfies the fourth preset condition, an instruction to turn on the scanning bed is initiated to move the bed in the out-bed direction, and an indication marker is displayed as an irregular shape 631 having an arrow and an arrow 632 pointing to the out-bed (OUT) of the scanning bed in the movement direction of the scanning bed; lastly, the user adds a finger to touch the touch screen, as shown by an indication marker 641 of the finger shown by 640 in FIG. 6, and moves the bed in the out-bed direction in an accelerated manner, at which time the irregular shape 631 with arrows remains unchanged, the arrow 632 pointing to the out-bed (OUT) of the movement direction of the scanning bed changes to a double arrow 642, and an indication marker 643 indicating acceleration (+Speed) is added. For more information about the indication marker, please refer to FIG. 4A-FIG. 4C and its related description.

It should be noted that the user's finger may or may not leave the touch screen 111 during an interval between the start command for indicating a movement of the scanning bed and the instruction for indicating a movement direction of the scanning bed.

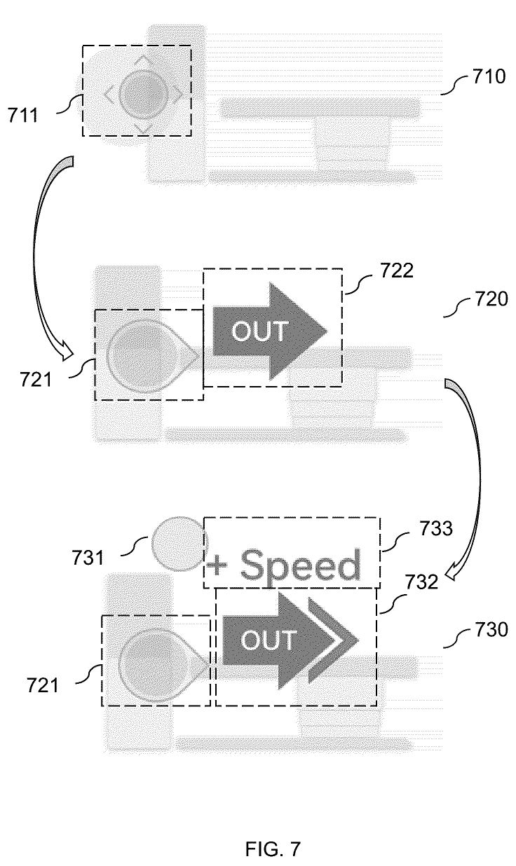

In some embodiments, the start instruction for starting moving the scanning bed and the instruction for indicating a movement direction of the scanning bed may be determined at the same time. For example, if the user touches the first position of the touch screen 111 with a touch pressure that satisfies the fourth preset condition and slides to the second position with a touch distance that satisfies the third preset condition, the start instruction for starting moving the scanning bed and the instruction for indicating a movement direction of the scanning bed may be determined simultaneously. As another example, as shown in FIG. 7 at 710, firstly, the user presses downwardly on the touch screen, and when the touch pressure satisfies the fourth preset condition, an indication marker is displayed as a circle 711; secondly, the user slides in the out-bed (OUT) direction, as shown in FIG. 7, and when the touch distance of the user satisfies the third preset condition, the instruction to turn on the scanning bed is initiated, and an indication marker is displayed as an irregular shape with an arrow 721 and an arrow 722 pointing to the out-bed (OUT) of the scanning bed in the movement direction of the scanning bed; lastly, a number or a count of fingers of the user that touches the touch screen is increased, which indicates an acceleration of the movement of the scanning bed. As shown by an indication marker 731 in FIG. 7, the movement in the out-bed direction is accelerated. At this time, the irregular shape 721 with arrows remains unchanged, the arrow 722 pointing to the out-bed (OUT) of the movement direction of the scanning bed changes to a double arrow 732, and an indication marker 733 indicating acceleration (+Speed) is displayed.

In some embodiments, when a number of touching fingers is greater than one, the processor 120 may accelerate the scanning bed 112 at a greater speed of scanning bed movement after the start instruction for starting moving the scanning bed and the instruction for indicating a movement direction of the scanning bed are determined. For more information about the movement speed of the scanning bed, please refer to the following description of FIG. 2. A larger movement speed of the scanning bed is a speed at which the scanning bed is moved when the number of touching fingers is greater than one. The larger movement speed of the scanning bed may be determined based on experience.

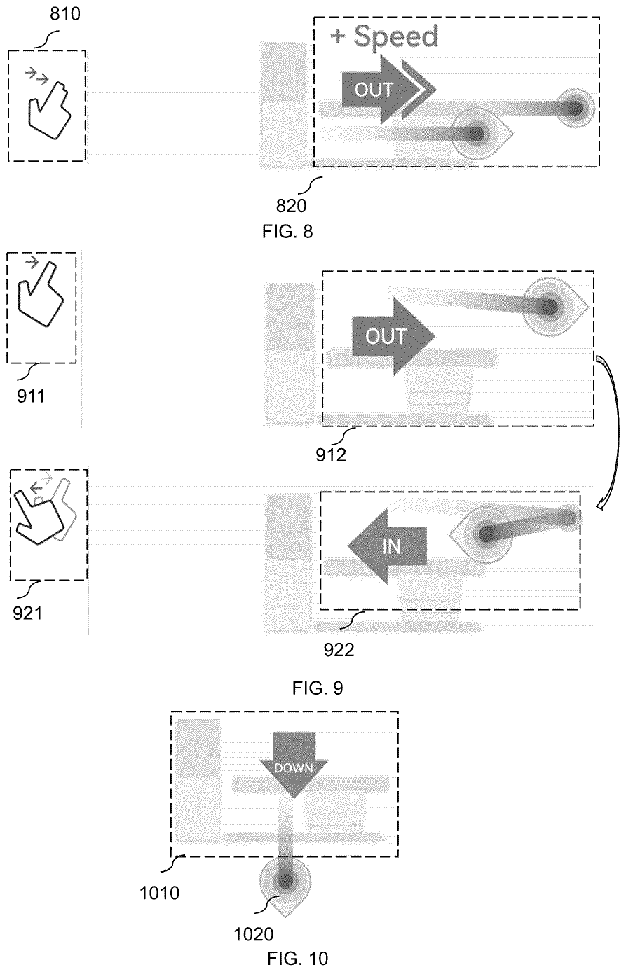

In some embodiments, when the user directly presses and slides downward with the number of touch fingers greater than 1, when the user's touch distance satisfies the third preset condition and at the same time the touch pressure satisfies the fourth preset condition, the processor may simultaneously generate the instruction for indicating a movement direction of the scanning bed, the start instruction for starting moving the scanning bed, and an instruction for accelerating a movement. As shown in FIG. 8, when the user directly presses and slides downward with 2 fingers(as shown in 810 in FIG. 8) when the touch distance satisfies the third preset condition while the touch pressure satisfies the fourth preset condition, the processor simultaneously generates the instruction for indicating a movement direction of the scanning bed, the start instruction for starting moving the scanning bed and the instruction for accelerating a movement, and performs a movement of the bed. An instruction marker is displayed as shown in 820 in FIG. 8.

In some embodiments, the instruction for accelerating a movement refers to a movement speed instruction that is temporarily configured to increase the movement speed of the scanning bed. The movement speed instruction refers to a base movement speed of the scanning bed. The base movement speed determines the movement speed of the scanning bed when the instruction for accelerating a movement is not used. In some embodiments, the instruction for accelerating a movement allows the scanning bed to increase the movement speed for a short period of time. The acceleration is typically temporary and is limited by the number of user fingers touching the screen or other mechanisms. The instruction for accelerating a movement typically takes effect on top of the movement speed instruction.

In some embodiments, the movement direction of the scanning bed 112 may be changed during movement. For example, after moving through the scanning bed 112 based on the movement direction, the user maintains touch with the touch screen 111 and the finger is offset to another movement direction (e.g., an original movement direction of the scanning bed 112 was out of the bed, moving from left to right, and the current user's finger is no longer sliding from left to right but instead is sliding from right to left). The processor 120 may take the original second position as a new first position and the position to which the current user's finger slides from right to left as a new second position, and when a touch distance between the new first position and the new second position satisfies the third preset condition, then the processor 120 may generate a new instruction for indicating the movement direction of the scanning bed 112, so as to realize a change in the movement direction of the scanning bed 112. When the touch distance between the new first position and the new second position does not satisfy the third preset condition, the movement direction of the scanning bed 112 is not changed. As another example, as shown in FIG. 9, during the process of the scanning bed being out of the bed, the finger slides from the left to the right as shown in FIG. 9 at 911, and an indication marker is displayed as shown in FIG. 9 at 912 (indicating that the movement direction is out of the bed (OUT)). When the finger no longer slides from the left to the right but from the right to the left (as shown in 921 in FIG. 9), and the touch distance from the right to the left satisfies the third preset condition, an instruction to change the movement direction of the scanning bed is turned on, and the scanning bed starts to move into the gantry, and an indication mark is displayed as shown in 922 in FIG. 9 (indicating the movement direction into the bed (IN)).

In some embodiments, the stop instruction for stopping moving the scanning bed is configured to control the stopping of movement of the scanning bed 112.

In some embodiments, the stop instruction for stopping moving the scanning bed is determined according that the touch screen 111 does not sense a touch operation within a target time period after the scanning bed has started moving. When the touch screen 111 does not sense a touch operation within the target time period, it is assumed that the user has stopped touching the touch screen 111, and the processor 120 generates the stop instruction for stopping moving the scanning bed.

The target time period is a specific time period. For example, the target time period may be 2 s, etc. In some embodiments, the target time period may be determined based on a touch operation. For example, the touch operation includes a double-click and a multi-click. When the user performs the double-click and the multi-click, there exists a time interval between two adjacent clicks, and the target time period may be slightly larger than the time interval. Thus, the processor 120 does not misjudge when generating the stop instruction for stopping moving the scanning bed and does not affect the sense of the user's experience due to the target time period being too long.

In some embodiments, during an actual bed moving process, the user may draw a preset graphic (e.g., “x”), a preset operation (e.g., continuously clicking on the screen twice or more than twice, etc.), or by clicking on the emergency stop icon on the touch screen, and the processor 120 may generate an emergency stop instruction upon detecting a preset graphic or a preset operation or the emergency stop icon and send the emergency stop instruction to the medical device to stop the movement of the scanning bed 112.

An instruction for indicating a target position that the scanning bed moves to is configured to control the scanning bed to move to a target position. The target position is a final position of the scanning bed after moving the bed. In some embodiments, an instruction for indicating a target position that the scanning bed moves to includes an instruction for indicating a horizontal target position that the scanning bed moves to, an instruction for indicating a vertical target position that the scanning bed moves to, etc.

In some embodiments, the instruction for indicating a horizontal target position that the scanning bed moves to is configured to control the scanning bed 112 to move to the horizontal target position.

In some embodiments, the vertical target position that the scanning bed moves to is used to control the scanning bed 112 to move to the vertical target position.

The horizontal target position is a position in the horizontal direction (in or out of the bed) that the scanning bed 112 needs to reach. The vertical target position is a position in the vertical direction (bed up and bed down) that the scanning bed 112 needs to reach. In some embodiments, after the start instruction for indicating a movement of the scanning bed is performed, the processor 120 may move the scanning bed to the horizontal target position or the vertical target position based on the slide of the user's finger. When the finger leaves the touch screen, the scanning bed stops moving.

In some embodiments, a total movement distance of the scanning bed in a certain movement direction is positively correlated with a touch parameter of the touch operation.

The total movement distance of the scanning bed is an actual distance moved by the scanning bed.

In some embodiments, the processor 120 may determine the total movement distance of the scanning bed in a variety of ways. For example, the greater the touch parameter (e.g., touch distance) of the touch operation is, the greater the total movement distance of the scanning bed is. As another example, the processor may determine by a preset table or vector database constructed based on historical data. The preset table/vector database may be a table or database characterizing a correspondence between different touch parameters and different total movement distances of the scanning bed. For more information about the touch parameter, please refer to FIG. 11 and its related description.

In some embodiments, the processor 120 may display, in real time, a total movement distance of the scanning bed and/or a target position of the scanning bed corresponding to the touch parameter, etc., in a display interface of the touch screen.

In some embodiments, different touch parameters, correspond to different total movement distances of the scanning bed. Different total movement distances of the scanning bed correspond to different positions of the scanning bed.

In some embodiments, the processor 120 may determine the total movement distance of the scanning bed based on the touch distance. For example, when the touch distance of the user's finger in a certain movement direction satisfies the third preset condition, the processor 120 determines the instruction for indicating a movement direction of the scanning bed and generates feedback for the instruction for indicating a movement direction of the scanning bed. After generating the instruction for indicating the movement direction of the scanning bed, the user's finger may leave the touch screen for a preset period of time, and after the user touches the touch screen 111 again and slides a preset distance in that movement direction again, the processor may capture the preset distance and determine a total movement distance of the scanning bed in that movement direction. The preset distance is a distance of sliding of the user's finger after touching the touch screen 111 again and performing a single-finger slide operation after determining the instruction for indicating a movement direction of the scanning bed. The preset time period may be determined based on manual input or a system default setting.

In some embodiments, when the touch distance of the user's finger in a certain movement direction satisfies the third preset condition, the processor 120 determines the instruction for indicating a movement direction of the scanning bed and generates feedback of the instruction for indicating the movement direction of the scanning bed. After generating the instruction for indicating the movement direction of the scanning bed, the user's finger may leave the screen of the touch screen within the preset period of time, and after the user touches the touch screen 111 again and slides a preset distance in that movement direction again, the processor may capture the preset distance and determine the total movement distance of the scanning bed in that movement direction. The preset distance is a distance of sliding of the user's finger for a single-finger slide operation after the user's finger touches the touch screen 111 again after determining the instruction for indicating the movement direction of the scanning bed.

When the touch distance of the user's finger in the certain movement direction satisfies the third preset condition, the finger does not leave the touch screen and continues to slide the preset distance along that movement direction. When the processor 120 senses the above touch operation, the processor 120 designates the direction along which the finger moves as the movement direction of the scanning bed, and at the same time determines the total movement distance of the scanning bed in that movement direction based on that preset distance.

In some embodiments, the preset distance of the user's finger slide is positively correlated with the total movement distance of the scanning bed. The greater the preset distance is, the greater the total movement distance of the scanning bed is.

In some embodiments, during the process of the user sliding the preset distance on the touch screen 111 to set the total movement distance of the scanning bed, the processor 120 may control the display interface to display in real time the total movement distance of the scanning bed or the target position of the scanning bed in a variety of ways (e.g., highlighting, coloring, bolding, blinking, etc.). For example, when the display interface presents an interface coordinate system, the processor 120 may detect in real time the preset distance of the user's finger sliding along a certain movement direction, control a synchronized movement of an executive subject in the corresponding direction in the interface coordinate system, and display the total movement distance of the scanning bed at the first preset position (e.g., below a real-time position of the executive subject or below a movement trajectory of the executive subject), and display a target position of the scanning bed at the second preset position (e.g., a corresponding position of the interface coordinate system), the corresponding position has a mapping relationship with the target position. For more information about the mapping relationship, please refer to the description below.

The executive subject is an image identifier of the whole comprising the scanning device, the patient and the scanning bed 112 in the display interface. The real-time position of the executive subject is a real-time position of the executive subject in the display interface.

In some embodiments of the present disclosure, the real-time display of the total movement distance with the scanning bed and/or the target position of the scanning bed allows the user to precisely control the movement of the scanning bed, thereby improving the efficiency and accuracy of the movement of the scanning bed. The user may adjust the position of the scanning bed based on the real-time displayed data to ensure that the scanning bed is moved to the target position.

In some embodiments of the present disclosure, a positive correlation between a movement distance of the scanning bed and a touch parameter of the touch operation may facilitate the user to accurately carry out the touch parameter, thereby improving the efficiency and accuracy of the bed moving.

In some embodiments, during the movement of the scanning bed 112, at least one of the movement speed, the position of the scanning bed, or the movement direction of the scanning bed 112 may be displayed in real time in the display interface of the touch screen.

The position of the scanning bed refers to a position of the scanning bed 112 during the moving process. For example, the position of the scanning bed may include information about the position of the scanning bed 112 in a physical space or information about a position of a scanning bed icon in the touch screen, etc. The physical space may refer to an environment in which the scanning bed 112 is located, such as an inspection room, a scanning room. The scanning bed icon refers to a graphical identification configured to represent the scanning bed 112 in the display interface.

In some embodiments, the position of the scanning bed may be represented by coordinates of a position of a point of the scanning bed 112 in a spatial coordinate system. The spatial coordinate system is a coordinate system of the physical space introduced to determine the position of the scanning bed. In some embodiments, the spatial coordinate system may be a three-dimensional right-angle coordinate system, e.g., a length direction of the scanning bed 112 may be the x-axis, a width direction of the scanning bed 112 may be the y-axis, a direction perpendicular to the scanning bed 112 may be the z-axis, and a coordinate origin may be a center of the scanning bed or any other arbitrarily specified point.

In some embodiments, the position of the scanning bed may be represented by coordinates of a position of a point of the scanning bed icon in an interface coordinate system, and the interface coordinate system is a coordinate system of a display interface introduced to determine the position of the scanning bed icon. In some embodiments, the interface coordinate system may be a two-dimensional right-angle coordinate system. For example, the interface coordinate system may have a length direction of the scanning bed 112 as the x-axis and a direction perpendicular to the scanning bed 112 as the y-axis, and a coordinate origin may be an initial position of the scanning bed. In some embodiments, the coordinate origin may also be a center of a scanning region, or may be any other arbitrarily specified point. The initial position is a position of the scanning bed before it begins to move. The scanning region is a region within which the medical device can scan a patient.

In some embodiments, the processor 120 may capture the position of the scanning bed 112 via various types of sensors before or during the scanning bed 112 begins to move. For example, the processor 120 may capture a position in the physical space where the scanning bed 112 is located in real time via a camera. As another example, the processor 120 may measure an exact coordinate and a distance of the scanning bed 112 in conjunction with a distance sensor.

In some embodiments, the processor 120 may obtain an image of an overhead view of the scanning bed 112 via a camera located above the scanning bed and in a fixed position. In some embodiments, the camera may be a 3D camera that may obtain a 3D image of the scanning bed 112 that is captured. In some embodiments, the camera may be a depth camera that may obtain depth information for photographing the scanning bed 112. The processor 120 may determine a position of the scanning bed based on the spatial coordinate system with the image. In some embodiments, the position of the scanning bed may be represented in the form of three-dimensional coordinates.

The real-time display refers to a synchronized movement of the position of the scanning bed icon in the display interface with the position of the scanning bed in the physical space. In some embodiments, the processor 120 may synchronize the movement by controlling the scanning bed icon in the display interface to move in real time based on the movement of the scanning bed 112 in the physical space. In some embodiments, the processor 120 may sample the movement of the scanning bed in the physical space based on the movement of the scanning bed in the physical space at a preset time interval, obtain a position of the scanning bed in the physical space, and control the movement of the scanning bed icon in the display interface to a corresponding position based on the position.

The position of the scanning bed icon refers to position information of the scanning bed icon in the display interface. For example, the position of the scanning bed icon may be represented by a positional coordinate of a point of the scanning bed icon in an interface coordinate system in the display interface.

In some embodiments, the process of the real-time display may be realized based on the spatial coordinate system of the scanning bed having a mapping relationship with the interface coordinate system. For example, during the process of performing movement of the scanning bed, the processor 120 may obtain a position of the scanning bed in the physical space (e.g., coordinates in the spatial coordinate system) in real time, map it to the interface coordinate system based on the mapping relationship, obtain a position of the mapped scanning bed icon (e.g., coordinates in the interface coordinate system), and control the movement of the scanning bed icon in the display interface to that position based on the position of the mapped scanning bed icon. In some embodiments, the mapping relationship may be a correspondence between coordinates in the spatial coordinate system of any point on the scanning bed 112 and coordinates in the interface coordinate system of a corresponding point on the display interface.

In some embodiments of the present disclosure, the synchronized display of the movement of the scanning bed through the display interface facilitates the user to observe and monitor the movement and parameters of the scanning bed, which further improves an interaction between the medical device and the user, and facilitates the user to use the medical device.

In some embodiments, the instruction for indicating a movement speed of the scanning bed is configured to control the movement speed of the scanning bed 112 for movement in the movement direction.

In some embodiments, the instruction for indicating a movement speed of the scanning bed is determined based on a number (or count) of touch traces separated from each other that are simultaneously obtained by the touch screen 111 during a detection time period.

The touch traces are touch traces of fingers when the user performs a touch operation on the touch screen 111 using the fingers. The number of touch traces may be determined based on a number of fingers of the user touching the touch screen 111. For example, when the user performs the touch operation using only one finger, the number of touch traces is 1. When the user performs the touch operation using two fingers, the number of touch traces is 2.

In some embodiments, a plurality of sets of movement speed combinations may be provided in correspondence for the medical device 110. Each set of movement speed combinations may refer to a combination of different movement speeds corresponding to a plurality of levels. Each set of movement speed combinations may include a plurality of levels of movement speeds.

In some embodiments, the processor 120 may adjust a movement speed combination currently used by the user based on a speed adjustment operation of the user at the medical device 110. For example, the user selects a movement speed combination 1 as the current movement speed combination. The user performs an accelerated movement during moving the bed using a certain level of the moving speed combination 1 while operating the medical device 110. If an operation frequency of the user performing the accelerated movement is greater than a frequency threshold, the processor 120 may increase the movement speed of that level in the movement speed combination 1 or switch a movement speed combination 2 to the current movement speed combination, the movement speed of the movement speed combination 2 as a whole is greater than the movement speed of the movement speed combination 1 as a whole.

In some embodiments, if the user, after switching the movement speed combination, still performs an accelerated movement when operating the medical device 110 and the operation frequency of the accelerated movement is greater than the frequency threshold, the processor 120 may repeat the above operation to increase the movement speed and determine a new movement speed combination until the user no longer performs the accelerated movement or the operation frequency of the accelerated movement is less than the frequency threshold, to satisfy the user's demand for the movement speed of the scanning bed, and thereby improving the interaction experience between the user and the medical device.

In some embodiments of the present disclosure, by means of multiple target control instructions regarding the scanning bed 112, the movement of the scanning bed 112 may be made accurate to meet the actual needs of the user, which is conducive to further improving the interaction experience between the user and the medical device 110.

In some embodiments, the movement speed of the scanning bed is positively correlated with the touch parameter of the touch operation.

In some embodiments, the touch parameter may include a number of touch fingers. The number of touch fingers is a number of fingers used by the user to perform the touch operation. When the number of touch fingers satisfies an acceleration condition, the processor 120 may determine a movement speed of the scanning bed in a variety of ways based on the touch parameter. For example, the movement speed of the scanning bed is positively correlated with the touch parameter (e.g., the touch pressure, the number of touch fingers, etc.). The greater the touch parameter is, the greater the movement speed of the scanning bed is.

In some embodiments, the movement speed of the scanning bed is positively correlated with a maximum touch pressure of the touch fingers. The greater the maximum touch pressure is, the greater the movement speed of the scanning bed is. In some embodiments, the movement speed of the scanning bed is positively correlated with an average value of touch pressures of all touch fingers. The greater the average value of the touch pressures is, the greater the movement speed of the scanning bed is. The maximum touch pressure is a maximum value of the touch pressure across all touch fingers.

In some embodiments of the present disclosure, based on the positive correlation between the movement speed of the scanning bed and the touch parameter, the user is facilitated to accurately adjust the movement speed of the scanning bed through the touch operation, so as to improve the accuracy and precision of moving the bed.

In some embodiments, the movement speed corresponding to the touch parameter is displayed in real time in the display interface of the touch screen.

In some embodiments, the processor 120 may control the display interface of the touch screen to display, in real time, a correspondence between the touch parameter and the movement speed of the scanning bed. The correspondence is used to reflect how the movement speed of the scanning bed varies with the touch parameter. The processor 120 may determine the correspondence between the touch parameter and the movement speed of the scanning bed based on a priori knowledge or historical data. For example, the correspondence may be a linear relationship or a curvilinear relationship.

In some embodiments, the processor 120 may display the correspondence between the touch pressure and the movement speed of the scanning bed in real time based on a mathematical model, a graph, or the like. For example, the processor 120 may establish a two-dimensional coordinate system with the touch pressure as the horizontal axis and the movement speed of the scanning bed as the vertical axis. The processor 120 may determine a linear relationship (e.g., a straight line, etc.) between the touch pressure and the movement speed of the scanning bed based on the touch pressure and the corresponding movement speed of the scanning bed at various moments by a fitting algorithm, and display it on the display interface. The fitting algorithm may include a least squares method, a Levenberg-Marquardt algorithm, a genetic algorithm, etc.

In some embodiments, the processor 120 may determine the movement speed of the scanning bed at various moments based on the maximum touch pressure or the average of the touch pressures of the touch fingers obtained in real time. The processor 120 may control a movement of a preset icon at various positions in the linear relationship based on the movement speed of the scanning bed at the various moments. The movement speed of the scanning bed at different moments corresponds to different positions of the preset icons on the linear relationship. The style of the preset icons may be customized as desired, such as presetting colors, sizes of the icons, or adding other visualization elements. In some embodiments, the processor 120 may control the touch screen to display the preset icon along with a numerical value for the movement speed of the scanning bed.

In some embodiments, the user usually needs to consider the speed of the scanning bed during moving the bed, so the processor may control the display interface to display only the movement speed of the scanning bed, avoiding the interference of other touch parameters to the user.

In some embodiments of the present disclosure, by displaying a correspondence between the touch parameter and the movement speed of the scanning bed in real time for use by the user to understand the pressure exerted by the finger on the touch screen, the adjustment of the movement speed of the scanning bed is facilitated to avoid the bed moving speed being too fast or too slow, and the accuracy and efficiency of the bed moving is improved.

In some embodiments, in response to a touch operation, the processor 120 may display a specified graphical user interface on the touch screen, for more information, please refer to FIGS. 4A-4C and its related description.

It should be noted that the present disclosure is also applicable to the control of other components of the medical device, for example, adjusting a distance between a detector at an upper end of a C-arm and a patient so as to bring a target imaging site of a target object (e.g., a lower limb, a heart, etc.) to a suitable scanning position, etc.

Step 230, for each of the at least one target control instruction, the processor 120 is configured to generate, via the touch screen, target sense feedback corresponding to the target control instruction from a plurality types of candidate sense feedback.

In some embodiments, each of the plurality of candidate control instructions may correspond to one of the plurality types of candidate sense feedback.

The target sense feedback is feedback given to the user through the touch screen 111 in response to the target control instruction. For example, the target sense feedback may be feedback on the success or failure of performing the target control instruction, feedback on a start instruction for moving the scanning bed, feedback on generating an instruction indicating a movement direction of the scanning bed, feedback on an instruction for accelerating a movement speed of the scanning bed, feedback on an instruction for stop moving the scanning bed, etc. In some embodiments, the processor 120 may generate the target sense feedback at least in the touch region of the touch screen 111 via a sense feedback technique. For example, the processor 120 may simulate a haptic experience of physical keys or different materials on the surface of the touch region through vibration, etc.

The touch region is a region where the user is in contact with the touch screen 111. For example, the region where the user's finger is in contact with the touch screen 111.

In some embodiments, the types of the candidate sense feedback may include one or more of tactile feedback (e.g., vibration feedback), visual feedback, auditory feedback, and the like. The tactile feedback may include, for example, vibration feedback. The tactile feedback (e.g., vibration feedback, etc.), the visual feedback, and the auditory feedback, etc., may each include a plurality of different types of candidate sense feedback. Each type of the candidate sense feedback of the plurality of the types of the candidate sense feedback may be in one-to-one correspondence with one of the plurality of candidate control instructions.

In some embodiments, the candidate sense feedback may include a vibration feedback generated by the touch screen 111 by way of vibration. In some embodiments, The vibration direction, the vibration intensity, the vibration sensation, the vibration frequency, the vibration duration, the vibration texture refer to relevant features of vibration feedback felt by the user when interacting with the touch screen. The vibration direction refers to a direction in which the vibration feedback is generated on the touch screen. The vibration direction may produce vibration sensations in different directions, for example, push or pull sensations. The vibration intensity refers to the strength or intensity of the vibration feedback of the touch screen. The vibration sensation is an overall feeling of the vibration feedback felt by the user through the touch screen. The vibration frequency is a rate or periodicity at which the vibration feedback is generated in the touch screen. Different types of vibration feedback differs in at least one of intensity, texture, duration, and vibration frequency.

FIG. 3B is an exemplary schematic diagram illustrating vibration feedback according to some embodiments of the present disclosure. The intensity refers to a magnitude of the vibration. The greater the intensity is, the stronger the vibration is and the more tactile the user feels. In some embodiments, the processor 120 may correspond different target control instructions to different intensities of vibration feedback based on actual needs. For example, vibration feedback of a greater intensity (e.g., a first intensity 301, a third intensity 303, etc., as shown in FIG. 3B) indicates a first-level target control instruction (e.g., a start instruction for starting moving the scanning bed, an instruction for indicating a movement speed of the scanning bed, an instruction for an abnormal failure, etc.). Vibration feedback of a weaker intensity (e.g., a second intensity 302, a fourth intensity 304, etc., shown in FIG. 3B) indicates a second-level target control instruction (e.g., an instruction indicating a movement direction of the scanning bed, etc.). The processor 120 may realize an adjustment of the intensity by adjusting an amplitude of a waveform, e.g., the higher the amplitude is, the higher the intensity is. The first-level target control instruction and the second-level target control instruction may be preset according to actual needs. For example, the first-level target control instruction and the second-level target control instruction may reflect different importance of the instruction.

The texture is a hardness of the vibration felt by the user. The harder the vibration (texture 1 as shown in FIG. 3B) is, the clearer and drier the texture felt by the user is. The rounder the vibration (Texture 2 as shown in FIG. 3B) is, the softer the texture felt by the user is. The processor may adjust the texture of the vibration by changing a curve of the waveform.

The duration is a duration of the vibration. A transient vibration (smaller than a duration threshold, e.g., duration of 0.01 seconds or less) may give the user clearer feedback. As shown in FIG. 3B, duration 2 is a transient vibration. A vibration with a longer duration (larger than a duration threshold, e.g., more than 0.5 seconds) may give the user stronger feedback. As shown in FIG. 3B, duration 1 is a longer duration vibration. In some embodiments, longer duration vibrations are generally not used except in urgent and important situations (e.g., when an error occurs, a warning, etc.).

The vibration frequency is a number of vibrations per second. As shown in FIG. 3B, frequency 1, frequency 2, and frequency 3 may represent low, medium, and high frequencies, respectively. For example, if a certain vibration feedback is a weak-to-strong vibration (e.g., a two-consecutive vibration, a three-consecutive vibration, etc.), the vibration felt by the user is more positive, and may correspond to feedback such as a certain target control instruction of the scanning bed 112 being performed successfully. As another example, if certain vibration feedback is from strong to weak vibration, the vibration felt by the user is more negative, and may correspond to feedback such as a certain target control instruction of the scanning bed 112 fails to be performed, e.g., when the scanning bed 112 reaches a boundary, a finger is moved out of the interface and cannot continue to move, etc. Exemplarily, as shown in FIG. 10, during the lowering of the bed of the scanning bed, the finger moves out of an interface 1010 and an indication marker 1020 of the finger is located outside of the interface 1010, which corresponds to vibration feedback of 2 vibrations with a shorter duration from strong to weak.

As another example, if certain vibration feedback is a medium-frequency vibration, the vibration felt by the user is neutral, and may correspond to the feedback that the touch pressure satisfies the start instruction determined by the fourth preset condition, etc. Different vibration motors correspond to different vibration frequencies (e.g., between 100 Hz and 200 Hz, etc.), which may be set according to actual needs. For example, a frequency of a pressure-sensitive vibration motor may be between 150 Hz and 180 Hz, to which the user's skin is most sensitive, without generating a sound that affects the user's sense of hearing.

In some embodiments, the processor 120 may determine a plurality of combinations including different intensities, different textures, different durations, and/or different vibration frequencies according to actual needs. Through the above combinations, different target control instructions and vibration feedback after execution of different target control instructions may be represented respectively. For example, a plurality of different combinations of different vibration frequencies and intensities may be used to clearly represent vibration feedback of different target control instructions.

In some embodiments, the processor 120 may control the duration of the standard vibration to be within 0.005 seconds and the frequency to be around 120 Hz. Based on the 4 dimensions of intensity, texture, duration, and vibration frequency, waveforms are fixed and combined through software to determine different vibration feedback.

In some embodiments, the processor 120 may set the vibration feedback according to actual needs, and not all of the target control instructions and feedback after the execution of the target control instructions need to be through the vibration feedback, but also through the visual feedback, the auditory feedback, etc.

In some embodiments, the vibration feedback may include at least one of vibration feedback of a movement state, vibration feedback of a movement direction, vibration feedback of a movement speed, or vibration feedback of an abnormal situation.

In some embodiments, the vibration feedback of the movement state corresponds to a start instruction for starting moving the scanning bed or a stop instruction for stopping moving the scanning bed. For example, when a user generates the start instruction for starting moving the scanning bed by a touch operation, the touch screen 111 emits the vibration feedback of the movement state after a touch pressure exerted by the user's finger satisfies the fourth preset condition.

In some embodiments, the vibration feedback of the movement direction corresponds to an instruction indicating a movement direction of the scanning bed. For example, when a user generates the instruction indicating a movement direction of the scanning bed by a touch operation, the touch screen 111 emits the vibration feedback of the movement direction after the user's finger slides to the second position where a touch distance from the first position satisfies the third preset condition.

In some embodiments, the vibration feedback of the movement speed corresponds to an instruction for indicating a movement speed of the scanning bed. For example, when a user generates an instruction for indicating a movement speed of the scanning bed by a touch operation, after the user touches the touch screen using two fingers, the touch screen emits the vibration feedback for the movement speed and vibration feedback for accelerating the movement speed.

In some embodiments, the vibration feedback of the abnormal situation corresponds to a situation in which a target control instruction is abnormal.

In some embodiments, a type of abnormality of the vibration feedback of the abnormal situation may include at least one of a movement failure, being out of a preset range of operation, etc. For example, when the user generates an instruction indicating a movement direction of the scanning bed by a touch operation, if a touch distance of the user's finger sliding from the first position to the second position of the touch screen 111 does not satisfy the third preset condition, the touch screen 111 emits the abnormal condition vibration feedback.

In some embodiments of the present disclosure, while the user generates a target control instruction through a touch operation, the finger is capable of receiving corresponding sense feedback instantaneously, and different information is conveyed through different tactile senses, which can enable the user to clearly perceive different information feedback (e.g., different movement directions, movement speeds of the scanning bed 112, boundary positions moved by the scanning bed, failure to move the bed, etc.) through the finger tactile senses without leaving the patient's line of sight, and further improve the interaction experience between the user and the medical device.

The auditory feedback may refer to auditory feedback information generated by the touch screen 111 by way of sound. For example, in response to movements in different directions of the scanning bed 112, the processor 120 may provide feedback via the touch screen of different sounds corresponding to movement directions. The user may determine a movement direction of the scanning bed through the auditory feedback. In some embodiments, the operation of starting or stopping moving the scanning bed, the movement speeds of the scanning bed, etc., may be fed back through voice.

In some embodiments, the sense feedback corresponding to different target control instructions may be determined by user customization or automatically by the system. The processor 120 may generate the sense feedback in a touch region of the touch screen 111 based on the different target control instructions and the sense feedback corresponding to the different target control instructions.

In some embodiments of the present disclosure, the processor 120 senses the touch operation via the touch screen 111, replacing traditional physical buttons, which can help the user to achieve a natural and labor-saving effect during the interaction with the medical device, and improve the interaction experience between the user and the medical device 110.

In some embodiments, each of the types of target control instructions corresponds to one type of touch operation, and the processor 120 may, for each type of target control instruction, determine a personalized touch region corresponding to the user based on historical touch operations of the user with respect to the target control instruction.

The types of target control instructions are one of a start instruction for starting moving the scanning bed or a stop instruction for stopping moving the scanning bed, an instruction for indicating a movement speed of the scanning bed, an instruction for indicating a target position that the scanning bed moves to, an instruction indicating a movement direction of the scanning bed, etc.

In some embodiments, different types of target control instructions correspond to different types of touch operations. The types of touch operations include one of an operation to move the bed, a rotation operation, etc. For example, a touch operation in which the user slides a first touch distance in a certain movement direction corresponds to the instruction for indicating a movement speed of the scanning bed; and a touch operation in which the user slides a second touch distance in a certain movement direction corresponds to the start instruction for starting moving the scanning bed. The operation to move the bed refers to translating the scanning bed in different directions to move the scanning bed to a desired position. The rotation operation refers to rotating the scanning bed along different directions to move the scanning bed to the desired position. The operation to move the bed may include at least one of in-bed direction, out-bed direction, raised-bed direction, lowered-bed direction, etc.

The historical touch operations are touch operations within a past time period. For example, the historical touch operations may include touch operations within a past week, a past month.

The personalized touch region refers to a region of touch operations determined based on user habits, needs, or preferences, etc. For example, the personalized touch region includes a position, a size, etc. of the touch region. Different users and different types of touch operations correspond to different personalized touch regions.

In some embodiments, the processor 120 may determine a personalized touch region corresponding to a user based on the user's historical touch operations of the target control instructions in various ways. For example, the processor 120 may count different users, and different kinds of touch operations based on historical data. For the same user and the same kind of touch operation, the largest touch region or the smallest touch region of that kind of touch operation is used as the personalized touch region of that user under that kind of touch operation.

The largest touch region or the smallest touch region may be a touch region with the largest area or the smallest area.

In some embodiments, the personalized touch region is determined based on historical touch operations of a user having the same type of touch operation as the touch operation.

In some embodiments, the processor 120 may determine, through process 231-process 232, a corresponding personalized touch region of a user having the same type of touch operation as the touch operation, based on the historical touch operations of that user.

Process 231, a corresponding reference touch distance is determined based on historical touch operations of a certain user and historical touch operations of a certain type.

The reference touch distance is a parameter value for determining a parameter value of a touch region corresponding to the touch operation.

In some embodiments, the processor 120 may determine the reference touch distance in a variety of ways. For example, the processor 120 may statistically analyze historical touch operations of different users and different types of touch operations to determine an average value of touch distances under a certain user and a certain type of touch operation as a reference touch distance of the user under that touch operation. Exemplarily, touch distances of a plurality of historical touch operations of a user A under an operation to move the bed are averaged; and an averaged result is used as a reference touch distance of the user A under the operation to move the bed. The average may be an arithmetic average or a weighted average, etc.

Process 232, based on the reference touch distance, with a shape of a preset touch region, the personalized touch region for the touch operation of the user in the type is determined.

The shape of the preset touch region is a preset shape of the touch region. For example, the shape of the preset touch region may be a circle, a rectangle, etc.

In some embodiments, the processor 120 may, in a variety of ways, determine a personalized touch region corresponding to a certain user under a certain type of touch operation, based on a reference touch distance of the user, of a certain type of touch operation, with the shape of the preset touch region. For example, when a touch operation of a certain user is an operation to move the bed, the processor 120 may construct a shape of the preset touch region as a personalized touch region of the user under the operation to move the bed based on the reference touch distance of the user under the operation to move the bed, in conjunction with an associated screen boundary, as the personalized touch region of the user under the operation to move the bed.

Exemplarily, assuming that the scanning device 113 is located on a left side of the scanning bed 112, the shape of the touch region is a rectangle, and the touch operation is an operation of moving the bed out of the bed, the user is required to slide from left to right on the touch screen, and the processor 120 may compose a rectangular personalized touch region based on the reference touch distance and the boundary of the touch screen. For example, the rectangular personalized touch region is constructed using the reference touch distance as a length of one side of the personalized touch region and a length of the associated screen boundary as a length of the other side of the personalized touch region. A position of the personalized touch region may be located between a start position of the touch operation and the associated screen boundary. The associated screen boundary is a boundary of the touch screen to which a movement direction of moving the bed points.

In some embodiments, the start position of the touch operation may be located on a boundary of the personalized touch region, or the start position of the touch operation may be located within the personalized touch region.

The start position of the touch operation is a position at which the user begins a touch operation on the touch screen.

In some embodiments, different users, different types of touch operations may correspond to a same preset shape of a touch region, or different users, different types of touch operations may each correspond to different preset shapes of touch regions. The processor 120 may obtain the shapes of the preset touch regions based on manual input or memory.

In some embodiments, the processor 120 may determine a personalized touch region of a certain user under a certain type of touch operation based on the processes 231-process 232. The processor 120 may also determine personalized touch regions under different users and different types of touch operations separately through the processes described above.

In some embodiments, when a user needs to switch to another type of touch operation after performing a certain type of touch operation, the processor 120 may call a personalized touch region corresponding to the other type of touch operation and control the touch screen to switch and display.

In some embodiments, when the personalized touch region corresponding to the other type of touch operation needs to be switched to, the processor 120 prompts the user through a variety of prompting methods. The prompting methods include, but are not limited to, visual, tactile, auditory, and other pathway methods. Exemplarily, the processor 120 may notify the user of the change in the touch region by providing a brief color-rendering reminder of the adjusted personalized touch region displayed on the touch screen, setting a new icon, a new color, etc. The processor 120 may provide a brief vibration or a vibration of other vibration frequencies to notify the user, the other vibration frequencies are vibration frequencies used to differentiate from other vibration feedback; or a sound alert via a speaker, etc.

In some embodiments of the present disclosure, for different types of touch operations, the user needs to touch the touch screen at different positions and sizes. By counting the historical touch operations of different users and different types of touch operations, and determining positions, sizes, and layouts of the touch regions, the users can be provided with more personalized operating experiences that are more in line with the operating habits of the users, improve the usability and ease of use of the display interface, and meet the specific needs of different users.

In some embodiments of the present disclosure, target control instructions such as determining a moving direction of the scanning bed require a user's finger to slide a certain distance. When a start position of the user's sliding is not selected properly, the user's touch operation may be caused to touch the touch screen boundary before it is completed (e.g., when sliding from left to right, the start position of the user's sliding is close to a right boundary of the screen, which causes the user's touch operation to touch the right boundary before it is completed, resulting in a failure of the touch operation). Thus the risk of a failed touch operation may be reduced by setting the personalized touch region.

It should be noted that the above description of the process 200 is merely for the purpose of exemplification and illustration, and does not limit the scope of application of the present disclosure. For those skilled in the art, various corrections and changes can be made to the process 200 under the guidance of the present disclosure. However, these amendments and changes remain within the scope of the present disclosure.

FIG. 4A is an exemplary schematic diagram illustrating a display of a specified graphical user interface on a touch screen according to some embodiments of the present disclosure. FIG. 4B is yet another exemplary schematic diagram illustrating a display of a specified graphical user interface on a touch screen according to some embodiments of the present disclosure. FIG. 4C is yet another exemplary schematic diagram illustrating a display of a specified graphical user interface on a touch screen according to some embodiments of the present disclosure.

In some embodiments, in response to a touch operation, the processor 120 may display a specified graphical user interface on the touch screen 111.

For more information about the touch screen 111, please refer to FIG. 1 and its related description. For more information about the touch operation, please refer to FIG. 2 and its related description.

The specified graphical user interface may refer to an interface that is available for a user to view and confirm. A size of the specified graphical user interface may be less than or equal to the size of the touch screen. For example, the specified graphical user interface may correspond only to a region where the user touches the touch screen 111 or to the entire touch screen 111. In some embodiments, the specified graphical user interface may include a virtual twin device of the medical device.

In some embodiments, different touch operations may correspond to different specified graphical user interfaces. There may be preset correspondences between the touch operations and the specified graphical user interfaces. When the user performs a certain touch operation, the processor 120 may display a specified graphical user interface corresponding to the touch operation on the touch screen for the user to view based on the above correspondence and the touch operation. As shown in FIG. 4A, when the user only touches the touch screen 111, the specified graphical user interface may include a first indication marker 401 corresponding to that touch region. As shown in FIG. 4B, when the user touches the touch screen 111 and makes a movement upward, if a touch distance of the user is greater than a distance threshold, the specified graphical user interface may include a second indication marker 402 characterizing a new touch region corresponding to that new touch region. The second indication marker 402 may represent a movement direction of a raised bed of the scanning bed 112. As shown in FIG. 4C, when the scanning bed 112 raises the bed, the movement speed is an accelerated movement speed when a number of touch traces of the user touching the touch screen 111 is greater than 1. The specified graphical user interface may include a third indication marker 403 characterizing the accelerated movement speed. For more information about the indication marker, please refer to the following related descriptions of FIGS. 4A-4C.

In some embodiments, in response to a touch operation, the processor 120 may display a specified graphical user interface on all or a portion of the touch screen 111.

In some embodiments of the present disclosure, in response to the touch operation, the processor 120 may display a specified graphical user interface on the touch screen 111 to enable a user to intuitively determine target control instructions and to improve the user's experience of interacting with the medical device.

In some embodiments, the specified graphical user interface may include a visual guidance region.

The visual guidance region refers to a region where a user may be visually guided. As described in the above embodiments, the second indication marker 402 in FIG. 4B has an upward protrusion that may serve as a visual guide to the user as to the movement direction of the raised bed of the scanning bed 112. A size of the visual guidance region may be the same as the size of the specified graphical user interface.

In some embodiments of the present disclosure, the visual guidance region may serve to visually guide the user in an intuitive manner.

In some embodiments, the visual guidance region may include a virtual twin device of the medical device and/or at least one control button, and the at least one control button corresponds to the at least one target control instruction.

In some embodiments, the virtual twin device of the medical device may refer to an image that simulates a relevant action of the medical device 110. The virtual twin device of the medical device may include a variety of constituent elements, for example, may include at least one of a simulated scanning bed, an indicator label, a display background, etc. The simulated scanning bed may refer to an associated icon that may represent the scanning bed 112.

The indication marker refers to an associated icon that may characterize a movement of the simulated scanning bed. The display background may refer to an underlying background of the virtual twin device of the medical device. The indication marker and the display background, etc., may be represented in a variety of ways, and the different representations may represent, respectively, whether the simulated scanning bed is moving and in different movement directions, etc. For example, the indication marker may be represented by different colors and/or shapes. The different colors may represent, respectively, that the simulated scanning bed is not moving or has different movement directions. The shapes of the indication marker may include a variety of shapes, for example, a circle, an irregular shape, etc. When the indication marker has no arrow (e.g., a circle), it may indicate that the simulated scanning bed is not moving, such as the first indication marker 401 shown in FIG. 4A. When the shape of the indication marker has an arrow (e.g., an irregular shape), a different direction of the arrow may indicate a different movement direction. The second indication mark 402, as shown in FIG. 4B, indicates a moving direction of the raised bed. The third indication mark 403, as shown in FIG. 4C, indicates an accelerated movement speed during the raising of the bed. As another example, the display background may be represented by different colors and/or textures. The different colors and/or textures may indicate the simulated scanning bed not moving or a different movement direction, etc., respectively.

In some embodiments, the indication marker may be generated based on a user's touch point on the touch screen. For example, when a user touches the touch screen, the processor may generate a corresponding touch point as an indication marker with no movement direction. When a touch distance of the user in a certain direction satisfies the third preset condition, the processor may generate the indication marker to indicate that moving direction. In some embodiments, the indication marker may be generated in a preset operation range based on the touch point. The preset operation range may be set based on actual needs.

In some embodiments, the virtual twin device of the medical device may be a static or dynamic twin image. The static twin image may characterize a movement direction of the simulated scanning bed by, for example, indication markers of different representations and/or display backgrounds. The dynamic twin image may characterize a movement direction of the simulated scanning bed by dynamic changes in the simulated scanning bed and/or the indication markers of the different representations and/or the display backgrounds.

Characterizing the virtual twin device of the medical device through the different representations of the indication markers and/or display backgrounds, etc., can make the visual feedback through the virtual twin device of the medical device graphic and concrete, and further improve the user's interaction experience with the medical device 110.

The control button refers to a key that allows for relevant control of the scanning bed 112. Multiple types of control buttons may correspond to different sets target control instructions, respectively. For example, the control buttons may include buttons indicating start, stop, different movement directions, different movement speeds, etc., of the movement of the scanning bed 112.

In some embodiments, the control buttons may include preset buttons A key, B key, etc.

The preset buttons A key, B key may refer to buttons that are preset in advance for scanning a certain part of the patient. For example, the A key may refer to scanning a head of the patient, the B key may refer to scanning a chest of the patient, etc. The preset buttons A key, B key, etc. may be set according to actual needs. The corresponding preset buttons A key, B key may be the same or different for different users. In some embodiments, the processor 120 may call up a setting key by a preset touch operation (e.g., touching the A key or the B key for 2 seconds, etc.). When the processor 120 senses through the touch screen 111 that a touch pressure of the user on the setting key satisfies a setting condition, the processor 120 may set a current position of the scanning bed 112 to correspond to scanning a certain part of the patient with the preset buttons A key or B key.

When a plurality of patients need to scan the same part, the user may directly move the scanning bed 112 to a position of a corresponding part of a patient by means of the above-described control buttons A key, B key, etc. Based on differences between different patients, fine-tuning is carried out, so that the user operates the scanning bed more naturally and with less effort, and repetitive operations are avoided, further improving the interaction experience between the user and the medical device 110.

In some embodiments of the present disclosure, characterizing different target control instructions by the virtual twin device of the medical device and/or multiple types of control buttons may make the target control instructions more intuitive and clear, further improving the interaction experience between the user and the medical device 110.

In some embodiments, the visual guidance region may include visual feedback of the target control instructions, the visual feedback may include multiple types, and each type of visual feedback may correspond to a set target control instruction, respectively.

The visual feedback may refer to visual feedback to a user based on the target control instructions.

The types of visual feedback may include a variety. In some embodiments, the visual feedback may include one or more of triggered movement visual feedback, movement direction visual feedback, and movement speed visual feedback, etc.

The triggered movement visual feedback may refer to feedback regarding target control instructions corresponding to the start instruction for starting moving the scanning bed or the stop instruction for stopping moving the scanning bed. The triggered movement visual feedback may indicate a start or stop state of the movement of the scanning bed 112. For example, the processor 120 may characterize the start or stop of the scanning bed 112 by 2 specified indication markers. When the target control instruction is the start instruction for starting moving the scanning bed or the stop instruction for stopping moving the scanning bed, the visual guidance region displays the above specified indication markers.

The movement direction visual feedback may refer to feedback regarding target control instructions corresponding to different movement directions of the scanning bed. The movement direction visual feedback may indicate a direction in which the scanning bed 112 is currently moving. For example, the second indication marker 402 may indicate that the scanning bed 112 is currently moving in the direction of the raised bed. In some embodiments, the processor 120 may generate the movement direction visual feedback via at least one of an arrow direction of the indication marker, a color change of the display background, and/or a texture change of the display background, etc.

The moving speed visual feedback may refer to feedback regarding target control instructions corresponding to different moving speeds of the scanning bed. The moving speed visual feedback may indicate a current speed at which the scanning bed 112 is moving. For example, the third indication marker 403 may indicate that the scanning bed 112 is currently moving at an accelerated speed during a process for raising the scanning bed 112. In some embodiments, the processor 120 may generate speed visual feedback based on a number of arrows of indication markers and/or a texture change in the display background. The second indication marker 402, 1 arrow, as shown in FIG. 4B, may indicate a preset default movement speed. The third indication marker 403, 2 arrows, as shown in FIG. 4C, may indicate an accelerated movement speed during the process for raising the scanning bed 112.

In some embodiments, before the instruction indicating a movement direction of the scanning bed is generated, the indication marker may indicate a plurality of movement candidate directions. After the instruction indicating a movement direction of the scanning bed is generated, the indication marker may indicate a movement direction of the scanning bed 112. In some embodiments, after the instruction to change a movement direction of the scanning bed is generated, the indication marker may display a changed movement direction of the scanning bed 112.

In some embodiments, after the instruction indicating a movement direction of the scanning bed is generated, the processor 120 may display a movement direction of the scanning bed 112 in the display background. In some embodiments, after the instruction indicating a movement direction of the scanning bed is generated, the processor 120 may change a color of the display background to a color corresponding to the movement direction of the scanning bed 112, etc.

In some embodiments, after the start instruction for starting moving the scanning bed is generated, the processor 120 may cause the color of the display background to change, or the texture of the display background to change, which in turn provides feedback on the start of the movement of the scanning bed 112.

In some embodiments of the present disclosure, different types of visual feedback through multiple ways may make the visual feedback more intuitive, more graphic, and more specific, further improving the interaction experience between the user and the medical device 110.

In some embodiments of the present disclosure, the display of target control instructions may be further made more intuitive and clear through multiple types of visual feedback of the target control instructions, further enhancing the interaction experience between the user and the medical device 110.

FIG. 5A is an exemplary flowchart illustrating a movement of a scanning bed according to some embodiments of the present disclosure. In some embodiments, process 500 may be performed by the processor 120.

In some embodiments, through a touch operation sensed by the touch screen, the processor may determine whether the scanning bed is moving, as well as determine a movement direction and a movement speed.

Step 511, the processor 120 is configured to sense, via the touch screen, whether a touch operation is existed. For more information, please refer to step 220 and its related description.

In response to No (the processor 120 senses via the touch screen 111 that no touch operation exists), step 512 is performed and no movement is performed.

The processor 120 senses by the touch screen 111 that no touch operation exists, the processor 120 may not generate a target control instruction and the scanning bed 112 may not perform any movement.

In response to Yes (the processor 120 senses by the touch screen 111 that the touch operation exists), step 513 is performed to determine whether the touch operation is in a touch control region.

In some embodiments, the touch screen 111 may be provided with the touch control region and a non-touch control region. The touch control region may refer to a user-specified touch operation region in the touch screen 111. When the user performs a touch operation in the touch control region, an instruction related to a movement of the scanning bed may be generated. When the touch operation is in the non-touch control region, the instruction related to the movement of the scanning bed cannot be generated. The processor 120 may provide feedback that the touch operation is in the non-touch control region via vibration feedback (e.g., vibration from strong to weak) of the touch screen 111 in order to alert the user that the touch operation is invalid, etc.

In response to Yes (the touch operation is in the touch control region), step 514 is performed to determine whether the touch distance satisfies the third preset condition.

When the touch operation is in the touch control region, the processor 120 may judge whether a touch distance of the user in a certain direction satisfies the third preset condition by the touch operation. For more information about the determining whether the touch distance satisfies the third preset condition, please refer to FIG. 2 and its related description.

When the touch distance does not satisfy the third preset condition, the processor 120 may not generate the target control instruction and the scanning bed 112 may not make any movement.

In response to Yes (the touch distance satisfies the third preset condition), step 515 is performed to determine the movement direction of the scanning bed.

When the touch distance satisfies the third preset condition, the processor 120 may determine a movement direction of the finger as a movement direction of the scanning bed 112, and prompt the user to the selected movement direction through the vibration feedback.

In response to Yes (the processor 120 senses via the touch screen 111 that the touch operation exists), step 521 is performed to determine a number of touch traces n.

In some embodiments, when the processor 120 senses via the touch screen 111 that the touch operation exists, during a movement of the scanning bed 112, the processor 120 may determine a number of touch traces and determine a movement speed.

In response to determining that the number of touch traces n is equal or less than a first threshold, step 522 is performed and the scanning bed moves at a constant speed.

In some embodiments, when the number of touch traces n is 1, the movement speed is a preset default movement speed, and the movement speed of the scanning bed 112 is unchanged.

In response to determining that the number of touch traces n is greater than the first threshold, step 523 is performed to increase the movement speed of the scanning bed.

In some embodiments, the movement speed is an accelerated movement speed when the number of touch traces n is greater than the first threshold (the vibration feedback may be a single strong vibration and/or a rounded vibration at a high frequency). The accelerated movement speed is greater than the default movement speed. For example, when the number of touch traces n is greater than the first threshold, a corresponding accelerated movement speed may be a fixed value regardless of the specific value of the number of touch traces. As another example, when the number of touch traces n is greater than the first threshold, the accelerated movement speed may be a plurality of values, and the specific value of the number of touch traces and the magnitude of the corresponding accelerated movement speed may be positively correlated.

FIG. 5B is an exemplary flowchart illustrating a movement of a scanning bed according to some embodiments of the present disclosure.

In some embodiments, the processor 120 may generate a target control instruction (a start instruction or a stop instruction) for controlling a movement of the scanning bed by a magnitude of a touch pressure sensed by the touch screen 111, which in turn enables a movement or a stop of the scanning bed 112.

In some embodiments, in response to determining that the touch pressure satisfies a second preset condition (e.g., the touch pressure being greater than a first pressure threshold (e.g., 2.5N, 3N, 3.5N, etc.)), the processor 120 may generate the start instruction for starting moving the scanning bed (corresponding vibration feedback may be a single strong vibration and/or a low-frequency rounded vibration sensation). The processor 120 may begin moving the scanning bed 112 based on the movement direction of the scanning bed 112 (e.g., determined in FIG. 5A). In some embodiments, the user's touch operation is uninterrupted, and the processor 120 may continue to perform the instruction to move the scanning bed. In some embodiments, the vibration feedback (e.g., 2 short vibrations from strong to weak, etc.) occurs when the scanning bed 112 moves to a boundary position (e.g., the highest position of the raised bed) or when the touch operation moves out of the touch control region, to remind the user that the touch operation is ineffective, then the scanning bed stops moving.

In some embodiments, a movement direction of the scanning bed 112 does not change during a movement of the scanning bed 112. In some embodiments, the movement direction of the scanning bed 112 may be changed during the movement of the scanning bed 112 in a manner that can be described in relation to FIG. 2.

In some embodiments, in response to determining that the pressure is less than a second pressure threshold (e.g., 0.1N, 0.2N, 0.5N, etc.), or the user's touch operation is interrupted, the processor 120 may generate the stop instruction for stopping moving the scanning bed.

In some embodiments, in response to determining that the touch pressure sensed by the processor 120 via the touch screen 111 is between a first pressure threshold and a second pressure threshold, the user's finger is disposed on the touch screen 111, performing a touch operation, the processor may perform steps 513 to 515 based on the touch operation. For more information about steps 513 to 515, please refer to FIG. 5A and its related description.

In some embodiments of the present disclosure, the user's interaction experience with the medical device 110 is enhanced by utilizing the user's sense of touch through a combination of pressure-sensing technology and sense feedback technology. In some embodiments of the present disclosure, the visual feedback and/or the auditory feedback may further enable the user to receive precise and intuitive feedback, and further enable the user to interact with the medical device 110 at a higher level. In some embodiments of the present disclosure, the pressure-sensing tactile (the combination of pressure-sensing technology and the sense-feedback technology) of the medical device 110 may make the user experience of the medical device 110 aligned with the user's familiar experience of C-suite products, and reduce the user's sense of experience disconnection.

FIG. 11 is an exemplary schematic diagram illustrating a process for generating at least one target control instruction according to some embodiments of the present disclosure.

In some embodiments, as shown in FIG. 11, the processor may obtain a touch threshold 1140; and when the touch operation 1150 satisfies a first preset condition 1160, the processor may generate a target control instruction 1170.

For more information about the touch operation, and the target control instruction, please refer FIG. 2 and their related descriptions.

The first preset condition 1160 is a condition to be satisfied by a touch parameter when a preset instruction indicating a movement direction of the scanning bed, a preset start instruction for starting moving the scanning bed, etc., is determined. In some embodiments, the first preset condition 1160 may be that a touch parameter of the touch operation is greater than the touch threshold 1140. The processor may preset the first preset condition according to actual needs.

The touch parameter is a parameter for describing a touch operation on the touch screen. For example, the touch parameter may include at least one of a touch position, a touch magnitude, a touch time, a touch pressure, a touch distance, a touch speed, etc. The touch position is a position that the user contacts on the touch screen. The touch magnitude refers to a magnitude of a portion of the touch screen that the user contacts on the touch screen, for example, a projected area of the touch portion on the screen. The touch time is a time for the user to perform a touch operation on the touch screen. The touch pressure is a pressure applied by the user when touching the screen. The touch distance is a distance that the user's finger moves on the touch screen. The touch speed is a speed at which the user moves his/her finger on the touch screen.

The touch threshold 1140 is a threshold that the touch parameter needs to meet. For example, the touch threshold 1140 may include a distance threshold 1141, a pressure threshold 1142, etc. The distance threshold 1141 is a minimum threshold value that the touch distance needs to satisfy. The pressure threshold 1142 is a minimum threshold that the touch pressure needs to satisfy.

In some embodiments, the touch threshold 1140 may be a system default value, a system preset value, or a value determined based on experimentation or experience. For more information about the touch threshold, please refer to FIG. 11 and its related description.

In some embodiments, the first preset condition 1160 may include a first touch distance in a certain movement direction being greater than a first distance threshold, and a second touch distance being greater than a second distance threshold. The first touch distance is a first distance at which the user's finger slides in a certain movement direction. The second touch distance is a distance at which the user finger continues to slide in the touch screen in a certain movement direction on the basis of the first distance. The second touch distance includes the first touch distance and the distance that the user finger continues to slide.

In some embodiments, the processor may determine a movement direction as a movement direction of the scanning bed upon determining that the touch distance is greater than the first touch threshold, and thus determine the instruction indicating a movement direction of the scanning bed. The processor may also determine to turn on the scanning bed when determining that the touch distance is greater than the second touch threshold, thereby determining an instruction to turn on the scanning bed.

The first distance threshold, the second distance threshold may be a system default value, a system preset value, etc., the second distance threshold is greater than the first distance threshold. In some embodiments, the second distance threshold may be set to a value slightly larger than the first distance threshold, which may avoid mis-operation of the user's finger.

In some embodiments of the present disclosure, by sensing the touch operation in real time and determining a relationship with the first preset condition, the user's touch operation may be quickly responded to, and a corresponding target control instruction may be determined, thereby improving the user experience. Avoiding bed moving caused by an improper operation or wrong operation such as the user's finger mistakenly touching the touch screen, the safety of the touch operation is improved.

In some embodiments, a real-time display of a current touch parameter of a current touch operation in relation to a magnitude of a touch threshold is displayed in the display interface of the touch screen.

For more information about the touch screen, the display interface, please refer to FIG. 1 and its related description.

The current touch operation is a touch operation sensed at a current time. The current time is a moment when a target control instruction of the scanning bed needs to be determined.

The current touch parameter is a touch parameter associated with the current touch operation. For example, the current touch parameter may include a current touch distance, a current touch pressure, etc.

In some embodiments, the processor may sense in real time the current touch parameter generated by the current touch operation corresponding to the user's finger on the touch screen at each moment, and display a magnitude of the current touch parameter of the current touch operation in relation to the touch threshold in a variety of ways. For example, the processor may use a visualization tool such as a chart to display the relationship between the current touch parameter and the touch threshold. For example, the processor may display a progress bar (e.g., a rectangular bar displayed horizontally or vertically). The progress bar may be divided into a plurality of sections (e.g., a current touch parameter, a first distance threshold, a second distance threshold) to indicate a completion degree of the touch operation. The processor may update the progress bar based on the touch parameter during the user's touch operation. For example, the processor may calculate a percentage of the touch parameter versus the touch threshold and update a corresponding portion of the progress bar to a completed state. The processor may integrate the designed progress bar with a display interface that is always visible during the period in which the user performs the touch operation, and update the progress bar in real time based on the touch parameter.

Exemplarily, the processor may create two adjacent rectangular boxes in order from smallest to largest, representing the first distance threshold, the second distance threshold, respectively; and create a rectangular box of the touch distance on a smaller side of the two adjacent rectangular boxes, and a length of the rectangular box of the touch distance dynamically varies according to the touch distance of the user's finger performing the touch operation at the touch screen. Initially, the length of the rectangular box of the touch distance should be 0 or very short, indicating that the touch operation has not started or has just started. While the touch operation is in progress, the processor periodically updates the state of the progress bar. For example, a percentage of the touch distance from the second distance threshold is calculated, and the length of the rectangular box of the touch distance is adjusted according to the calculated percentage. When the touch distance is greater than the second distance threshold, the rectangular box of the touch distance completely fills the rectangular box of the first distance threshold, the second distance threshold.

In some embodiments, the processor may render the rectangular box of the first distance threshold, the second distance threshold, and the touch distance, respectively, through a variety of visual effects, such as a gradient, an animation, or a color change, to attract the user's attention.

In some embodiments, the processor may display a progress bar of the touch pressure and the pressure threshold in a similar manner to the display of touch distance.

In some embodiments of the present disclosure, displaying the magnitude of the current touch parameter in relation to the touch threshold in real time may facilitate enhancing the user's experience feeling and improving the user's satisfaction with use. For example, during the real-time display, the user may understand the distance between his or her touch operation and the distance threshold through real-time feedback, adjust his or her touch operation (e.g., touch pressure, touch distance) in time, and improve the operation efficiency.

In some embodiments, the processor may obtain a personalized touch threshold corresponding to a user performing the touch operation.

The personalized touch threshold is a threshold for reflecting a feature of the user's own touch operation. For example, the personalized touch threshold includes a personalized distance threshold, a personalized pressure threshold, a personalized speed threshold, etc., of the user.

In some embodiments, as shown in FIG. 11, the touch threshold 1140 includes a personalized touch threshold. The personalized touch threshold may include a personalized distance threshold 1141 and a personalized pressure threshold 1142, etc.

In some embodiments, the processor may determine the personalized touch threshold in a variety of ways. For example, the processor may preset correspondences between different users and different personalized touch thresholds, and determine the personalized touch threshold by looking up a table. The correspondence may be determined based on historical data.

In some embodiments of the present disclosure, the personalized recognition experience may be improved by setting the personalized touch threshold.

In some embodiments, as shown in FIG. 11, the processor determines a personalized touch threshold of a corresponding user based on a hand feature 1110 of the user corresponding to the touch operation 1150.

The hand feature of the user refers to a physical feature of the user's hand. For example, the hand feature of the user may include a size of the user's finger, a flexibility degree of the finger, etc. The size of the user's finger is used to reflect a size/contour of the user's finger, for example, the size of the user's finger includes a diameter of the user's finger. The flexibility degree of the user's finger is used to refer to a coordination and agility of the user in manipulating, using, or controlling the user's finger.

In some embodiments, the processor may determine the hand feature of the user corresponding to the touch operation in a variety of ways. For example, the processor may capture an image of the user's hand via a camera, and the hand image is used to obtain the size of the user's finger bellies via an image recognition algorithm. The hand image is an image including a hand of the user. The image recognition algorithm includes, but is not limited to, a convolutional neural network, a support vector machine, etc. As another example, the processor may capture a video of the hand through a camera, determine a time for the user to complete a preset action, and/or determine a similarity degree between the completed action and the preset action. The processor may determine the flexibility degree of the finger through a first preset correspondence based on different time of completing the preset action, the similarity degree between the completed action and the preset action. The first preset correspondence may represent a correspondence among different time of completing the preset action, different similarity degrees of the completed action and the preset action, and different flexibility degrees of different fingers. The preset action may include a preset finger sliding route etc. The completed action refers to a route produced by an actual sense operation performed by the user's finger on the touch screen. The hand video is a video of the finger performing the preset action.

In some embodiments, the processor may determine the similarity degree between the completed action and the preset action in a variety of ways. For example, the processor may determine the similarity degree between the completed action and the preset action based on a Pearson correlation coefficient, Euclidean distance, etc., of the completed action and the preset action.

In some embodiments, as shown in FIG. 11, the personalized touch threshold may include a personalized distance threshold 1141.

The personalized distance threshold is a distance threshold corresponding to each of the different users.

In some embodiments, the processor may determine a personalized distance threshold of a user based on a hand feature of the user in a variety of ways. For example, the processor may determine the personalized distance threshold based on the hand feature of the user via a second preset correspondence. Exemplarily, the second preset correspondence may include: the greater size of the corresponding finger of the user is, the larger the personalized distance threshold corresponding to the user is; and the worse the flexibility degree of the corresponding finger of the user is, the larger the personalized distance threshold corresponding to the user is.

In some embodiments, second preset correspondences between hand features of different users and different personalized distance thresholds may be determined based on historical data or a priori knowledge.

In some embodiments of the present disclosure, the personalized recognition experience may be improved by setting the personalized touch threshold (e.g., the personalized distance threshold). For different users, biometric features may have subtle differences. For example, a size of a user's finger, a flexibility degree of a finger, and so on, may vary from person to person. By setting a unique personalized distance threshold for each user, a biometric feature of a user may be captured more accurately, thereby improving the accuracy and reliability of the sense-touch operation; and through the setting of the personalized distance threshold, a better balance between a false recognition rate and security, thus improving the security of the system.

In some embodiments, the personalized touch threshold may include a personalized pressure threshold In some embodiments, as shown in FIG. 11, the processor may determine a pressure average value 1130 based on pressure data 1120 of historical touch operations of the user; and based on the pressure average value 1130, determine the personalized touch threshold 1140 of the corresponding user. The personalized pressure threshold is a pressure threshold corresponding to each of the different users.

For more information about the historical touch operations, please refer to FIG. 2 and its related description.

The pressure data is a measurement of the touch pressure during the historical touch operations. For example, the pressure data may include measurement data of touch pressures within a past week, or a past month.

In some embodiments, the processor may, in a variety of ways, determine a pressure average value and, based on the pressure average value, determine a personalized pressure threshold for a corresponding user. For example, the processor may determine pressure average values of different users based on identity information of the different users, and determine the personalized pressure thresholds based on pressure average values according to a predetermined rule. The predetermined rule may refer to a process or an algorithm for determining the personalized pressure thresholds based on the pressure average value. Exemplarily, the predetermined is that the personalized pressure threshold is positively correlated with the pressure average value, and the greater the pressure average value is, the greater the personalized pressure threshold is.

The identity information refers to a variety of information reflecting identity features of a user, e.g., the identity information may include any one or more of: face recognition information, living body recognition information (iris recognition information, fingerprint recognition information, etc.) of the user. In some embodiments, the processor may obtain the identity information in a variety of ways. For example, the processor may obtain the identity information of the user through any one or a combination of a camera configured in the medical device, a fingerprint collector, etc.

In some embodiments, the processor may statistically analyze pressure data of the user in a touch screen operating system, determine a pressure average value of the pressure data, and determine a personalized pressure threshold based on the pressure average value according to the predetermined rule.

The touch screen operating system may be any device or instrument used by a user that is capable of recording touch pressures. In some embodiments, the touch screen operating system may be integrated or included in the medical device 110. Exemplary touch screen operating system may include at least one of an operating room device, medical diagnostic device, etc.

In some embodiments of the present disclosure, an individualized touch threshold (e.g., the personalized pressure threshold) is determined by a pressure average value, the user's use of the touch screen may be better reflected through the user's pressure average value, facilitating targeted determination of the personalized pressure thresholds for different users to improve the user's experience.

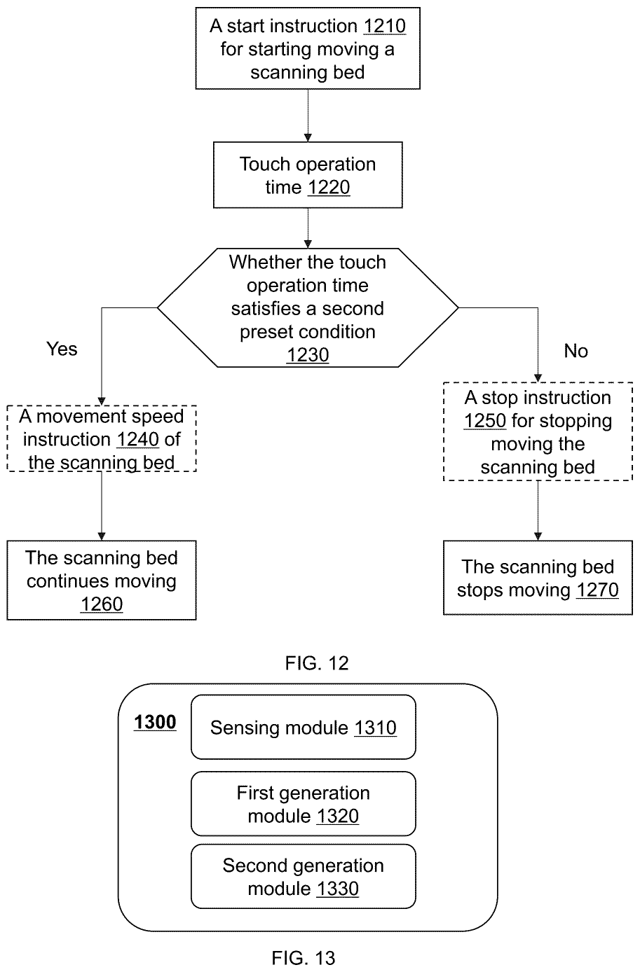

FIG. 12 is an exemplary schematic diagram illustrating a process for determining a touch operation time according to some embodiments of the present disclosure.

In some embodiments, as shown in FIG. 12, whether no touch operation is sensed on a touch screen is determined; in response to determining that no touch operation is sensed on the touch screen, whether a touch operation time satisfies a second preset condition 1230 is determined; the scanning bed is controlled to continue moving from an initial moving state in response to determining that the touch operation time satisfies the second preset condition 1230; or the scanning bed is controlled to stop moving from the initial moving state in response to determining that the touch operation time does not satisfy the second preset condition 1230.

The start instruction is an instruction for triggering the scanning bed to start moving. For more information about the start instruction, please refer to FIG. 2 and its related description.

For more information about the touch operation, please refer to FIG. 2 and its related description.

The touch operation time may refer to a sustained duration of the last touch operation by the user's finger when no touch operation is detected on the touch screen. For example, if a time point when no touch operation is detected on the touch screen is time point A, and a start time point of a most recent history touch operation before the time point A is time point B, then the touch operation time is an interval time between the time point A and the time point B.

The second preset condition is a judgment condition for evaluating whether the scanning bed stops or continues moving. For example, the second preset condition may include the touch operation time exceeding a time threshold. The time threshold may be a system default value, a system preset value, etc.

In some embodiments, as shown in FIG. 12, in response to the start instruction 1210 being turned on, the user's finger continues to slide on the touch screen, and when the touch distance satisfies a first preset condition, the processor may generate an instruction indicating a movement direction of the scanning bed to trigger the scanning bed 112 to move. The first preset condition may refer to a relevant condition for determining the instruction indicating the movement direction of the scanning bed. For example, the first preset condition includes that the touch distance is greater than a distance threshold. During the movement of the scanning bed 112, the processor may continuously capture the touch operation of the user's finger through the touch screen and count the touch operation time 1220 of the touch operation. When the user's hand finger stops the touch operation, the processor detects no touch operation of the touch screen, and determines whether the touch operation time satisfies the second preset condition 1230; in response to the touch operation time exceeding a time threshold, indicating that the user needs to continue moving the scanning bed 112, a movement speed instruction 1240 of the scanning bed is generated based on a similar manner as in FIG. 2, controlling the scanning bed continues moving 1260; in response to the touch operation time for which the time threshold is not exceeded, indicating that the user does not need to continue moving the bed, a stop instruction 1250 for stopping moving the scanning bed is generated, controlling the scanning bed stops moving 1270.

In some embodiments, when the user's finger stops the touch operation, the scanning bed continues moving without stop, the processor may notify the user that the scanning bed continues moving by prompting the user via a display interface (e.g., a text, a voice announcement, a preset specific icon, etc.) or by some other means (e.g., preset vibration feedback) to ensure consistency between the user's wishes and the results of the operation.

For more information about the instruction indicating the movement direction of the scanning bed, the instruction for indicating the movement speed of the scanning bed, and the stop instruction for stopping moving the scanning bed, please refer FIG. 2 and their related descriptions.

For more information about the touch distance, the first preset condition, please refer to FIG. 11 and the related descriptions thereof.

In some embodiments, during an actual movement of the bed, a user may draw a preset graphic (e.g., “x”), a preset operation (e.g., continuously clicking on the screen twice or more than twice), or by clicking on an emergency stop icon on the touch screen, etc., the processor may generate an emergency stop instruction upon detecting the above-described operation (e.g., the preset graphic, the preset operation, the emergency stop icon, etc.), and send the emergency stop instruction to the medical device to stop the movement of the scanning bed.

In some embodiments of the present disclosure, by counting the touch operation time, an interruption of the bed moving process may be avoided when the user's finger leaves the touch screen, ensuring the continuity of the bed moving process. By prompting the user through the display interface, the user's wishes and the results of the operation are kept consistent, and the efficiency and accuracy of the bed moving are improved.

Some embodiments of the present disclosure provide a control device (hereinafter referred to as a control device) of a medical device, a touch screen for sensing a touch state or a touch-release state of at least one position on the touch screen; a vibration element that is connected with the touch screen and for generating vibrations to generate sense feedback corresponding to target control instructions; and a processor configured to generate the target control instructions of the medical device in response to the touch state or the touch-release state of the touch screen, and to control the vibration element to generate vibrations.

In some embodiments, the control device is detachably provided or fixedly provided on a housing of the medical device.

In some embodiments, a touch operation in the touch state includes at least one of a single click, consecutive multi-click, a simultaneous multi-click, sliding from a first position of the touch screen to a second position of the touch screen.

In some embodiments, the processor may further be configured to obtain a touch threshold; and in response to determining that the touch operation satisfies a first preset condition, generate the at least one target control instruction, the first preset condition including that a touch parameter of the touch operation is greater than the touch threshold.

In some embodiments, the processor may also be configured to display a real-time comparison between a current value of the touch parameter of the touch operation and the touch threshold on a display interface of the touch screen.

In some embodiments, the processor may further be configured to obtain a personalized touch threshold corresponding to a user performing the touch operation.

In some embodiments, the processor may further be configured to determine the personalized touch threshold based on a hand feature of the user.

In some embodiments, the processor may further be configured to determine a pressure average value based on pressure data of historical touch operations of the user; and determine the personalized touch threshold corresponding to the user based on the pressure average value.

In some embodiments, the medical device includes a scanning bed, and the at least one target control instruction includes at least one of a start instruction for starting moving the scanning bed, a stop instruction for stopping moving the scanning bed, an instruction for indicating a movement speed of the scanning bed, an instruction for indicating a target position that the scanning bed moves to, or an instruction indicating a movement direction of the scanning bed.

In some embodiments, the movement speed of the scanning bed is positively correlated with a touch parameter of the touch operation.

In some embodiments, the control display module may further be configured to display, in real time, the movement speed corresponding to the touch parameter on a display interface of the touch screen during a movement of the scanning bed.

In some embodiments, the control display module may further be configured to display, in real time, a position of the scanning bed on a display interface of the touch screen during a movement of the scanning bed.

In some embodiments, the processor may further be configured to: determine whether no touch operation is sensed on a touch screen; in response to determining that no touch operation is sensed on the touch screen, determine whether a touch operation time satisfies a second preset condition; control the scanning bed to continue moving from the initial moving state in response to determining that the touch operation time satisfies the second preset condition; or control the scanning bed to stop moving from the initial moving state in response to determining that the touch operation time does not satisfy the second preset condition.

In some embodiments, a total movement distance of the scanning bed is positively correlated with a touch parameter of the touch operation.

In some embodiments, the processor may also be configured to display, in real time on a display interface of the touch screen, the total movement distance and/or the target position of the scanning bed corresponding to a current value of the touch parameter.

In some embodiments, the target control instructions include a plurality of types, each target control instruction corresponding to a touch operation; the processor may further be configured to, for each target control instruction, determine a personalized touch region corresponding to the user based on historical touch operations of the user with respect to the target control instruction.

In some embodiments, the personalized touch region is determined based on the historical touch operations of the user with the same type as the touch operation.

In some embodiments, the sense feedback includes a plurality of types, the target control instructions include a plurality of types, and each target control instruction corresponds to a type of sense feedback.

In some embodiments, the processor may further be configured to: display a specified graphical user interface on the touch screen in response to the touch operation.

In some embodiments, the specified graphical user interface includes: a visual guidance region.

In some embodiments, the visual guidance region includes: a virtual twin device of the medical device and/or at least one control button, and the at least one control button corresponds to the at least one target control instruction.

In some embodiments, the visual guidance region includes visual feedback of at least one type. The visual feedback of each of the at least one type corresponds to one of the at least one target control instruction.

For more information about the control device of the medical device, please refer to the previous related description.

FIG. 13 is an exemplary module diagram illustrating a system for controlling a medical device according to some embodiments of the present disclosure.

In some embodiments, a control system 1300 of a medical device may include: a sensing module 1310, a first generation module 1320, and a second generation module 1330.

In some embodiments, the sensing module 1310 may be configured to sense a touch operation via a touch screen, the touch operation being performed by a user on the touch screen.

In some embodiments, in response to the touch operation, the first generation module 1320 may be configured to generate, from a plurality of candidate control instructions, at least one target control instruction for controlling the medical device.

In some embodiments, for each of the at least one target control instruction, the second generation module 1330 may be configured to generate, via the touch screen, target sense feedback corresponding to the at least one target control instruction from a plurality types of candidate sense feedback, each of the plurality of candidate control instructions corresponding to one of the plurality types of candidate sense feedback.

In some embodiments, a touch operation includes at least one a single click, consecutive multi-click, a simultaneous multi-click, sliding from a first position of the touch screen to a second position of the touch screen.

In some embodiments, the first generation module 1320 may also be configured to obtain a personalized touch threshold corresponding to a user performing the touch operation. In some embodiments, the first generation module 1320 may also be configured to determine a personalized touch threshold of a corresponding user based on a hand feature of the user corresponding to the touch operation.

In some embodiments, the first generation module 1320 may further be configured to determine a pressure average value based on pressure data of historical touch operations of the user; and determine the personalized touch threshold corresponding to the user based on the pressure average value.

In some embodiments, the medical device includes a scanning bed, and the at least one target control instruction includes at least one of a start instruction for starting moving the scanning bed, a stop instruction for stopping moving the scanning bed, an instruction for indicating a movement speed of the scanning bed, an instruction for indicating a target position that the scanning bed moves to, or an instruction indicating a movement direction of the scanning bed.

In some embodiments, the movement speed of the scanning bed is positively correlated with a touch parameter of the touch operation.

In some embodiments, the control display module may further be configured to display, in real time, the movement speed corresponding to the touch parameter on a display interface of the touch screen during a movement of the scanning bed.

In some embodiments, the control display module may further be configured to display, in real time, a position of the scanning bed on a display interface of the touch screen during a movement of the scanning bed.

In some embodiments, a total movement distance of the scanning bed is positively correlated with a touch parameter of the touch operation.

In some embodiments, the control display module may also be configured to display, in real time on a display interface of the touch screen, the total movement distance and/or the target position of the scanning bed corresponding to a current value of the touch parameter.

In some embodiments, the touch operation is performed by a user in a personalized touch region of the touch screen, and the personalized touch region corresponds to the user and a type of the touch operation.

For more information about the control system 1300 of the medical device, please refer to the previous related description.

It should be understood that the control system 1300 of the medical device and its modules shown in FIG. 13 may be implemented utilizing a variety of approaches. It should be noted that the above description of the control device 1300 of the medical device and its modules is provided for descriptive convenience only and does not limit the present disclosure to the scope of the embodiments cited. It can be understood that for those skilled in the art, with an understanding of the principle of the system, it may be possible to make any combination of modules or form subsystems to be connected to other modules without departing from this principle. In some embodiments, the sensing module 1310, the first generation module 1320, and the second generation module 1330 disclosed in FIG. 13 may be different modules in a single system, or a single module may implement the functions of two or more of the modules described above. For example, the individual modules may share a common storage module, and the individual modules may each have a respective storage module. Variations such as these are within the scope of protection of the present disclosure.

The present disclosure provides a non-transitory computer-readable storage medium, comprising a set of instructions, when performed by a computer, the set of instructions cause the computer to perform a method including: sensing a touch operation via a touch screen, the touch operation being performed by a user on the touch screen; in response to the touch operation, generating, from a plurality of candidate control instructions, at least one target control instruction for controlling the medical device; and for each of the at least one target control instruction, generating, via the touch screen, target sense feedback corresponding to the target control instruction from a plurality types of candidate sense feedback, each of the plurality of candidate control instructions corresponding to one of the plurality types of candidate sense feedback.

The basic concepts have been described above, apparently, in detail, as will be described above, and does not constitute limitations of the disclosure. Although there is no clear explanation here, those skilled in the art may make various modifications, improvements, and modifications of present disclosure. This type of modification, improvement, and corrections are recommended in present disclosure, so the modification, improvement, and the amendment remain in the spirit and scope of the exemplary embodiment of the present disclosure.

1. A method for controlling a medical device implemented on a processing device including one or more processors and one or more storage media, comprising:
sensing a touch operation via a touch screen, the touch operation being performed by a user on the touch screen;
in response to the touch operation, generating, from a plurality of candidate control instructions, at least one target control instruction for controlling the medical device; and
for each of the at least one target control instruction, generating, via the touch screen, target sense feedback corresponding to the target control instruction from a plurality types of candidate sense feedback, each of the plurality of candidate control instructions corresponding to one of the plurality types of candidate sense feedback.

2. The method of claim 1, wherein the touch operation includes at least one a single click, a consecutive multi-click, a simultaneous multi-click, sliding from a first position of the touch screen to a second position of the touch screen.

3. The method of claim 2, further comprising displaying a real-time comparison between a current value of a touch parameter of the touch operation and a touch threshold on a display interface of the touch screen.

4. The method of claim 2, further comprising:
determining a pressure average value based on pressure data of historical touch operations of the user; and
determining a personalized touch threshold corresponding to the user based on the pressure average value.

5. The method of claim 1, wherein the medical device includes a scanning bed, and the at least one target control instruction includes at least one of a start instruction for starting moving the scanning bed, a stop instruction for stopping moving the scanning bed, an instruction for indicating a movement speed of the scanning bed, an instruction for indicating a target position that the scanning bed moves to, or an instruction for indicating a movement direction of the scanning bed.

6. The method of claim 5, wherein the movement speed of the scanning bed is positively correlated with a touch parameter of the touch operation, the touch parameter is a parameter for describing a touch operation on the touch screen.

7. The method of claim 5, further comprising: controlling a movement of the scanning bed based on the at least one target instruction.

8. The method of claim 7, further comprising: displaying, in real time, at least one of a movement speed, a position of the scanning bed, or a movement direction of the scanning bed on a display interface of the touch screen during the movement of the scanning bed.

9. The method of claim 7, further comprising: synchronizing the movement of the scanning bed by controlling a scanning bed icon in a display interface to move in real time based on the movement of the scanning bed in a physical space.

10. The method of claim 5, wherein the at least one target control instruction is the start instruction for starting moving the scanning bed, and the method further comprises:
determining whether no touch operation is sensed on the touch screen;
in response to determining that no touch operation is sensed on the touch screen, determining whether a touch operation time satisfies a second preset condition;
in response to determining that the touch operation time satisfies the second preset condition, controlling the scanning bed to continue moving from an initial moving state; or
in response to determining that the touch operation time does not satisfy the second preset condition, controlling the scanning bed to stop moving from the initial moving state.

11. The method of claim 5, wherein a total movement distance of the scanning bed is positively correlated with a touch parameter of the touch operation.

12. The method of claim 11, further comprising:
displaying, in real time on a display interface of the touch screen, the total movement distance and/or the target position of the scanning bed corresponding to a current value of the touch parameter.

13. (canceled)

14. (canceled)

15. The method of claim 1, wherein the plurality types of candidate sense feedback are different in at least one of a vibration direction, a vibration intensity, a vibration sensation, or a vibration frequency.

16. The method of claim 15, comprising:
displaying a specified graphical user interface on the touch screen in response to the touch operation.

17. The method of claim 16, wherein the specified graphical user interface includes: a visual guidance region.

18. The method of claim 17, wherein the visual guidance region includes: a virtual twin device of the medical device and/or at least one control button, and the at least one control button corresponds to the at least one target control instruction.

19. The method of claim 17, wherein the visual guidance region includes: visual feedback of at least one type, the visual feedback of each of the at least one type corresponds to one of the at least one target control instruction.

20. A system for controlling a medical device, comprising:
a touch screen for sensing a touch state or a touch-release state of at least one position on the touch screen;
a vibration element that is connected with the touch screen and for generating vibrations to generate target sense feedback corresponding to at least one target control instruction; and
a processor configured to generate the at least one target control instruction of the medical device in response to the touch state or the touch-release state of the touch screen, and to control the vibration element to generate vibrations.

21. The system of claim 20, wherein the control device is detachably provided or fixedly provided on a housing of the medical device.

22. The system of claim 21, wherein a touch operation in the touch state includes at least one of a single click, a consecutive multi-click, a simultaneous multi-click, sliding from a first position of the touch screen to a second position of the touch screen.
